(19) United States
(12) Patent Application Publication
(10) Pub. No.: US 2019/0214136 A1
(43) Pub. Date: Jul. 11, 2019

(54) PREDICTIVE BIOMARKERS OF DRUG RESPONSE IN MALIGNANCIES

(71) Applicant: Regents of the University of Minnesota, Minneapolis, MN (US)

(72) Inventors: Brian Van Ness, Minneapolis, MN (US); Monica Kieffer Akre, Minneapolis, MN (US); Wen Wang, Minneapolis, MN (US)

(21) Appl. No.: 16/032,660

(22) Filed: Jul. 11, 2018

Related U.S. Application Data
(60) Provisional application No. 62/531,207, filed on Jul. 11, 2017.

(51) Int. Cl. G16H 50/20; C12Q 1/6827; G16H 10/40; G16H 10/60; G16H 50/50; G16H 50/30; G16H 50/70; G16B 50/20; G16B 40/30; G16B 25/10; G16B 20/20

(52) U.S. Cl. CPC G16H 50/20; C12Q 1/6827; G16H 10/40; G16H 10/60; G16H 50/50; G16B 20/20; G16H 50/70; G16B 50/20; G16B 40/30; G16B 25/10; G16H 50/30

(57) ABSTRACT

In some examples, the techniques and systems described herein relate to generating a patient-specific drug-response prediction. For example, the technique may involve generating a drug-response expression profile corresponding to an efficacious therapeutic effect of a drug based on analysis of gene-expression and drug-response data stored in a database for one or more cell lines. The technique may also involve comparing a gene-expression profile of a patient tumor to the drug-response expression profile in order to generate a report of a patient-specific drug-response prediction. The report of the patient-specific drug-response prediction may identify one or more drugs likely to have an efficacious therapeutic effect on the patient tumor.

This application claims the benefit of, and priority to, U.S. Provisional Patent Application Ser. No. 62/531,207, entitled “PREDICTIVE BIOMARKERS OF DRUG RESPONSE IN MALIGNANCIES” and filed on Jul. 11, 2017, the entire content of which is incorporated by reference herein.

### BACKGROUND

Personalized medicine includes the identification of the biomarkers and/or expression profiles important in the treatment of cancer. Cancer cell lines offer a representative diversity of molecular processes involved in both malignant phenotypes as well as therapeutic responses. Coupling cancer line expression with high-throughput drug screens allows for examination of perturbed pathways that influence drug sensitivities.

### SUMMARY

Despite improvements in cancer therapies, wide variation in tumor response to treatment is a major limitation in achieving consistent therapeutic effect. The variation is likely represented by tumor diversity. Computational approaches are described to define response and resistance based on gene expression profiles in myeloma, a plasma cell malignancy, as one example cell type. This approach, as described with respect to the example of myeloma, takes advantage of a large panel of myeloma cell lines representing a wide diversity of response to therapeutic drugs used in the clinic.

Numerous collections of cancer cell lines provide additional opportunities to characterize the genetic signatures that may distinguish response and resistance to a variety of drugs. The NCI-60, Cancer Cell Line Encyclopedia (CCLE), and the Genomics of Drug Sensitivity in Cancer (GDSC) provide a wealth of information that includes genomic sequences, mutational status, gene expression, as well as response to panels of drugs used in cancer therapies. This offers a unique opportunity to apply approaches that may provide genetic profiles that define response and resistance, with the potential to apply these as predictors in clinical decisions and to improve patient outcomes. The NCI-60, CCLE, and GDSC, or other collections of cell lines also may provide opportunities to characterize genetic signatures that may distinguish response and resistance to a variety of drugs indicated for conditions other than cancers. In addition, such cell lines may provide opportunities to characterize genetic signatures that may distinguish other attributes (e.g. phenotypes) of the cell lines that may have other health- and wellness-related, scientific, aesthetic, or other significance. Therefore, the systems and techniques described herein are applicable to determining gene expression profiles for any type of cell and using such gene expression profiles to identify potential therapies directed to gene expression of each cell type.

In one example, a system comprises: a database coupled to a communication network and configured to store gene-expression data for a plurality of cell lines and drug-response data indicating a response of each cell line of the plurality of cell lines to a drug; processing circuitry coupled to the database and configured to: receive, from a remote computer, a gene-expression profile of a patient tumor; receive, from the remote computer, a request for a report of a drug-response prediction of the patient tumor to the drug, and, in response to the request: generate a drug-response expression profile based on the gene-expression data for the plurality of cell lines and the drug-response data, wherein the drug-response expression profile comprises a pattern of gene expression corresponding to an efficacious therapeutic effect of the drug; compare the gene-expression profile of the patient tumor to the drug-response expression profile; determine whether the gene-expression profile of the patient tumor is substantially similar to the drug-response expression profile; produce the report of the drug-response prediction; and transmit the report of the drug-response prediction to the remote computer.

In another example, a method comprises: receiving, by a database coupled to a communication network, gene-expression data for a plurality of cell lines and drug-response data indicating a response of each cell line of the plurality of cell lines to a drug; and by processing circuitry coupled to the database: receiving a gene-expression profile of a patient tumor from a remote computer; receiving a request for a report of a drug-response prediction of the patient tumor to the drug from the remote computer and, in response to the request: generating a drug-response expression profile based on the gene-expression data for the plurality of cell lines and the drug-response data, wherein the drug-response expression profile comprises a pattern of gene expression corresponding to an efficacious therapeutic effect of the drug; comparing the gene-expression profile of the patient tumor to the drug-response expression profile; determining whether the gene-expression profile of the patient tumor is substantially similar to the drug-response expression profile; producing the report of the drug-response prediction; and transmitting the report of the drug-response prediction to the remote computer.

In another example, a system comprises: means for receiving gene-expression data for a plurality of cell lines and drug-response data indicating a response of each cell line of the plurality of cell lines to a drug; means for receiving a gene-expression profile of a patient tumor from a remote computer; means for receiving a request for a report of a drug-response prediction of the patient tumor to the drug from the remote computer and, in response to the request: generating a drug-response expression profile based on the gene-expression data for the plurality of cell lines and the drug-response data, wherein the drug-response expression profile comprises a pattern of gene expression corresponding to an efficacious therapeutic effect of the drug; comparing the gene-expression profile of the patient tumor to the drug-response expression profile; determining whether the gene-expression profile of the patient tumor is substantially similar to the drug-response expression profile; producing the report of the drug-response prediction; and transmitting the report of the drug-response prediction to the remote computer.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1A-1F are graphical representations of example data gathering and analysis techniques in accordance with the examples of this disclosure.

FIGS. 2A-2C are graphical representations of example data analysis techniques in accordance with the examples of this disclosure.

FIG. 3 is a graphical representation of an example data analysis technique in accordance with the examples of this disclosure.

FIG. 4 is a graphical representation of an example data analysis technique in accordance with the examples of this disclosure.

FIG. 5 is a graphical representation of an example data analysis technique in accordance with the examples of this disclosure.

FIG. 6 is a graphical representation of an example data analysis technique in accordance with the examples of this disclosure.

FIG. 7 is a graphical representation of an example data analysis technique in accordance with the examples of this disclosure.

FIG. 8A-8F are graphical representations of an example data gathering and analysis technique in accordance with the examples of this disclosure.

FIG. 9 is a graphical representation of an example data analysis technique in accordance with the examples of this disclosure.

FIG. 10 is a graphical representation of an example data analysis technique in accordance with the examples of this disclosure.

FIG. 11 is a graphical representation of an example data analysis technique in accordance with the examples of this disclosure.

FIGS. 12A and 12B are graphical representations of an example data analysis technique in accordance with the examples of this disclosure.

FIGS. 13A-13C are graphical representations of an example data analysis technique in accordance with the examples of this disclosure.

FIG. 14 is a graphical representation of an example data analysis technique in accordance with the examples of this disclosure.

FIGS. 15A and 15B are graphical representations of an example data analysis technique in accordance with the examples of this disclosure.

FIGS. 16A-16D are graphical representations of an example data analysis technique in accordance with the examples of this disclosure.

FIG. 17 is a graphical representation of an example data analysis technique in accordance with the examples of this disclosure.

FIG. 18 is a graphical representation of an example data gathering and analysis technique in accordance with the examples of this disclosure.

FIG. 19 is functional block diagram illustrating an example system that may be used to implement the techniques described herein, which may include remote computing devices, such as a server and one or more other computing devices, that are connected to one or more external devices via a network.

FIG. 20 is a functional block diagram of the example external server in the example system of FIG. 19 that may be used to implement the techniques described herein.

FIG. 21 is a flow diagram illustrating an example technique for generating a patient-specific drug-response prediction in accordance with the examples of this disclosure.

### DETAILED DESCRIPTION

In general, this disclosure describes example systems and techniques related to predicting a drug response that a patient is expected to have to a particular drug prior to an actual response of the patient to the drug, such as prior to administration of the drug to the patient. A predicted drug response may be identified in a report, generated by processing circuitry coupled to a communication network, which may indicate whether the drug is expected to have an efficacious therapeutic effect or a non-efficacious effect on the patient.

The processing circuitry may generate the report of the drug-response prediction in response to receiving a gene-expression profile of patient tissue affected by a disease or condition (e.g., tissue from a patient tumor) and a request for the report of the drug-response prediction of the patient tissue to the drug from a remote computer, which may be accessed by a clinician, a healthcare professional, or other user. The processing circuitry may, in some examples, also generate the gene-expression profile. In other such examples, the processing circuitry may generate the report of the drug-response prediction in response to receiving a gene-expression profile of known cell lines (e.g., cell lines on which gene-expression information is stored in a database such as GDSC). Although reports of drug-response predictions are described herein as being generated by processing circuitry in response to receiving a gene-expression profile, the processing circuitry may be configured to produce reports containing other information in response to receiving a gene-expression profile. For example, the processing circuitry may be configured to produce a report of a predisposition or current expression of a patient tissue or other cell line of a disease, condition, or other phenotype in response to receiving the gene-expression profile. In any such examples, the processing circuitry may store the gene-expression profile and/or the report of the drug-response prediction or predisposition in a memory for further analysis or future reference.

In any such examples, a gene-expression profile may indicate expression levels of one or more genes expressed by cells of the patient tissue or other cell line. In examples in which the processing circuitry is configured to produce a report of the drug-response prediction for a patient, the processing circuitry may retrieve, from a database coupled to a communication network, gene-expression data for a plurality of cell lines and drug-response data indicating a response of each cell line of the plurality of cell lines to the drug. The processing circuitry then may generate a drug-response expression profile (e.g., a pattern of gene expression corresponding to an efficacious therapeutic effect of the drug) based on the gene-expression data for the plurality of cell lines and the drug-response data and compare the gene-expression profile of the patient tissue to the drug-response expression profile. Based on whether the gene-expression profile of the patient tissue is substantially similar to the drug-response expression profile, the processing circuitry then may produce the report of the drug-response prediction. The drug-response prediction may indicate whether the drug is expected to have an efficacious therapeutic effect or a non-efficacious effect on the patient tissue. In some examples, an efficacious therapeutic effect of the drug may be one or more of a reduction in a size of a patient tumor, a change in one or more biomarkers indicative of a disease status of the patient, or a reduction in one or more symptoms experienced by the patient.

Based on the drug-response prediction, a clinician may determine whether or not to administer the drug to the patient. For example, if the drug-response prediction indicates that the drug is expected to have an efficacious therapeutic effect on the patient tissue, the clinician may administer the drug to the patient. In other examples, if the drug-response prediction indicates that the drug is not expected to have an efficacious therapeutic effect on the patient tissue, the clinician may determine not to administer the drug to the patient. In such other examples, the clinician (or another user of a system including the processing circuitry) may submit a request for a report of a drug-response prediction of the patient tissue to a second drug to the system including the processing circuitry.

The processing circuitry then may generate a report of the drug-response prediction of the patient tissue to the second drug, which may indicate whether the second drug is expected to have an efficacious therapeutic effect on the patient tissue. For example, the processing circuitry may receive, from a remote computer, a request for a report of a second drug-response prediction of the patient tumor to a second drug, responsive to the request, retrieve, from the database, gene-expression data for a second plurality of cell lines and drug-response data indicating a response of each cell line of the second plurality of cell lines to the second drug, and generate a second drug-response expression profile based on the gene-expression data for the second plurality of cell lines and the drug-response data indicating a response of each cell line of the second plurality of cell lines to the second drug. The second drug-response expression profile may be a pattern of gene expression corresponding to an efficacious therapeutic effect of the second drug on the patient tumor or other cell line, or a non-efficacious effect of the second drug on the patient tumor or other cell line. In some examples, the processing circuitry may produce the report of the second drug-response prediction by comparing the gene-expression profile of the patient tumor to the second drug-response expression profile, determining whether the gene-expression profile of the patient tumor is substantially similar to the second drug-response expression profile, and producing the report of the second drug-response prediction. In some examples, the processing circuitry also may store the report of the second drug-response prediction in a memory and/or transmit the report of the second drug-response prediction to a remote computer. The processing circuitry may perform this process for several drugs for each of one or more gene expression profiles for respective cell types.

If the report of the drug-response prediction of the patient tissue to the second drug indicates that the drug is expected to have a therapeutic effect, the clinician may administer the second drug to the patient. If the report of the drug-response prediction of the patient tissue to the second drug indicates that the drug is not expected to have a therapeutic effect, the clinician or other user of the system may continue to submit one or more additional requests for reports of drug-response predictions of the patient tissue to one or more additional drugs. In addition, the system may enable a clinician to determine which drug or drugs is likely to have a therapeutic effect on patient tissue that previously responded to a particular drug but that no longer responds to the drug.

In any such examples, the system may enable a clinician to determine which drug or drugs is likely to have a therapeutic effect on the patient tissue prior to administering (e.g., prescribing) a particular drug to the patient, and thereby avoid administering a drug that is unlikely to have an efficacious therapeutic effect on the patient tissue. Avoiding the administration of drugs unlikely to have an efficacious therapeutic effect may provide one or more benefits. For example, avoiding the administration of such drugs may help improve the patient's clinical outcome by more promptly providing efficacious therapy in examples in which the patient's condition or disease is progressive in nature, as may be the case with many types of cancer. Prompt administration of efficacious therapy may enable treatment of cancer at a relatively earlier stage, such as prior to metastasis, thereby increasing a likelihood that the patient may achieve remission, a reduction of the number, severity, or duration of symptoms, or other clinical benefits. In addition, prompt administration of efficacious therapy may reduce inefficiencies associated with administering drugs that do not provide an efficacious therapeutic effect, and/or may enable the patient to avoid experiencing undesired effects associated with a drug that are not offset by the provision of efficacious therapeutic effect.

Other techniques for selecting a drug to administer to a patient to treat a particular disease or condition may rely on a trial-and-error approach. For example, a clinician may select at random a drug indicated for the disease or condition of the patient. In other examples, the clinician may select a drug indicated for the disease or condition of the patient that the clinician believes may provide efficacious therapeutic effects based on one or more criteria, such as overall efficacy of a drug among a population of patients having the same disease or condition. However, such approaches are inefficient and may result in the clinician administering one or more drugs to the patient that ultimately do not provide efficacious therapeutic effect. In some examples, such as examples in which the patient's disease or condition is expected to progress rapidly and/or is at an advanced stage, the delay caused by the administration of a drug that does not provide efficacious therapeutic effect may result in undesired clinical outcomes. In any such examples, the delay caused by the administration of a drug that does not provide efficacious therapeutic effect may result in the patient experiencing undesired effects associated with ineffective or undesirable drug selection and, in some examples, unnecessary monetary cost (e.g., the cost of the drug and/or other related costs of medical care). Thus, the trial-and-error approach of selecting a drug to administer to a patient may be inadequate for some patients. In contrast to this trial-and-error approach, the techniques described herein may generate and/or receive gene expression profile of the specific cell(s) in question and generate a drug-response prediction based on the gene expression profile to identify drugs likely to be effective in treating the condition of the patient

In some example techniques described herein, processing circuitry of a system may identify biomarkers and/or expression profiles common to cell lines (e.g., cancer cell lines) that respond to, or do not respond to, a particular drug. Based on the identified biomarkers and/or expression profiles, the processing circuitry may determine a drug-response expression profile common to cell lines that respond to, or do not respond to, the drug. As discussed herein, the term “responder” may be used to describe a cell line on which a drug has an efficacious therapeutic effect. Similarly, the terms “non-responder” may be used herein to describe a cell line on which a drug does not have an efficacious therapeutic effect. Alternatively, a cell line on which a drug does not have an efficacious therapeutic effect may be described herein as having “resistance” to the drug.

Drug response coupled with gene expression data through publicly-available sources may be used as a tool to determine what probesets are most valuable in determining response or resistance. Described herein is a computational pipeline that uses RNA sequencing (RNAseq) data to create gene expression signatures associated with cell line response or resistance, as well as an example application of these signatures in predicting therapeutic response in myeloma clinical trials. In some examples, gene expression profiles of cell lines may be used after specific drug resistance has emerged to identify signatures of response to other drugs, which may provide more effective therapeutic options.

The example techniques and systems for drug-response prediction described herein may be used to predict whether a drug will have an efficacious therapeutic effect on cells having a particular gene-expression profile (e.g., cells derived from a patient tumor). Such techniques may be used for virtually any disease or condition that may be treated with one or more drugs, such as types of cancers and other conditions. In addition, such techniques may be used to characterize genetic signatures that may distinguish other attributes (e.g., phenotypes) of a cell line that may have other health- and wellness-related, scientific, aesthetic, or other significance. One example technique involves generating, via processing circuitry coupled to the database, a drug-response gene expression profile of cell lines that have been shown to respond to a drug, based on the gene-expression and drug-response data stored in the database. A drug-response expression profile may include a pattern of expression of one or more genes that is common to each of the cell lines. For example, the drug-response expression profile may identify one or more genes that each of the cell lines expresses at a corresponding particular level. Such one or more genes may be considered a biomarker for cell response to the drug. Thus, identification of the presence of the biomarker in a gene-expression profile of a sample cell line may indicate that the sample cell line also will respond to the drug.

Such techniques may be repeatedly carried out to identify one or more additional drugs to which cells of tissue from which the sample cell line was derived (e.g., a patient tumor) may respond, either prior to beginning treatment with a first drug or thereafter. For example, such techniques may be repeated if the tissue from which the cell line was derived ceases to respond to a particular drug, which may indicate that a gene-expression profile of the cell line has changed, such as may occur due to ongoing mutation of the DNA of the cell line. The example techniques described herein thus may be used to predict whether cells of the tissue will respond to a drug and may facilitate one or more interventions associated with a favorable clinical outcome of the patient.

For the sake of illustration, the example techniques described herein are described within the context of an example application of a technique that may be carried out using the Sanger Institute's Genomics of Drug Sensitivity in Cancer database (GDSC) to develop a bioinformatic and pathway analysis approach that identifies the BCR pathway as an important biomarker that may be used in predicting cell response to the tyrosine kinase inhibitor, dasatinib, used in hematologic cancer therapies. This approach establishes a process by which data from cell line repositories can be used to identify biomarkers associated with drug response in the treatment of cancers. However, this approach also may be used to identify biomarkers that may indicate the response to other drugs of cell lines corresponding to other types of diseases or conditions, and should not be understood to be limited to the BCR pathway or to hematologic cancer therapies. For example, any of the techniques and systems described herein may be used with the GDSC, or any other suitable database, to identify biomarkers corresponding to other pathways, types of cancers, or other conditions. In addition, although the examples described herein are described with respect to human patients or portions thereof (e.g., human genetic material, expression products, cells, and/or tissues), they are not so limited. In alternative examples, the examples described herein may be implemented in non-human patients, e.g., non-human primates, canines, felines, equines, pigs, ovines, bovines, and rodents. These other animals may undergo clinical or research therapies that may benefit from the subject matter of this disclosure.

The GDSC is a collection of 1,047 cell lines from diverse tumor types that have been tested with 265 drugs. The data collection includes DNA sequence, mutation status, and gene expression data that may enable development of a pipeline of computational approaches that predict response and resistance. As described herein, drug response of the cell lines to dasatinib may be determined by a 9-step 2-fold serial dilution of drug concentration and measuring cell viability. From these, two quantitative values are provided: the drug concentration sufficient to reduce cell viability by 50% (IC50) and the area under the dose-response survival curve (AUSC). Gene expression data is available from the Affymetrix U219 gene array platform. Using the GDSC database, differentially expressed drug-response gene signatures may be used to identify profiles of cell lines and tumors that may respond to an individual therapy, such as a particular drug. These data may be used, such as by processing circuitry of a computer in communication with the database, to extract gene expression signatures of response and resistance to selected drugs across the GDSC B-cell hematologic malignancy-derived cell lines. Initial analyses may identify differential expression signatures of the responder versus the non-responder lines for an expanding number of drugs, which may inform the development of prediction algorithms capable of identifying response in cell lines as of yet untreated (e.g., cell lines for which drug-response data is not yet stored in the database) and in patient clinical trials.

Because expression patterns may vary widely simply based on tissue specificity, some example techniques may focus on data pertaining to cell lines that correspond to a similar disease or condition as a disease or condition that corresponds to a sample cell line. For example, the example technique described below is described with respect to B-cell malignancies, represented in the GDSC by 71 cell lines derived from leukemias, lymphomas, and myelomas. In other examples, however, some example techniques may evaluate drug responses of a larger group of cell lines, such as each of the 1,047 cell lines in the GDSC.

The example technique described below comprises a series of steps in which processing circuitry coupled to a communication network and the GDSC may determine drug response or drug resistance of a plurality of B-cell lines, develop a differential classification profile of gene-expression patterns (e.g., a drug-response expression profile), identify features by pathway analysis, and validate data (e.g., gene signatures) on cell lines and reported clinical outcomes. In the example described below, this approach to stratify and predict drug response is described in the context of the protein tyrosine kinase inhibitor drug dasatinib. However, this example should not be considered limiting, as the approach described with respect to dasatinib may be applicable to validating gene signatures associated with response to other drugs and/or other phenotypes. Dasatinib is a multi-target kinase inhibitor that has affinity for about 50 kinases and is most widely used to manage chronic myelogenous leukemia. Dasatinib's ability to reversibly and competitively inhibit the ATP binding site of kinases make its potential applications wide in scope. However, therapeutic applications have been reported in multiple cancers with significant variation in response. The example technique described below provides an approach to identifying a 5-gene signature distinguishing dasatinib response and resistance among the 71 cell lines derived from leukemias, lymphomas, and myelomas represented in the GDSC.

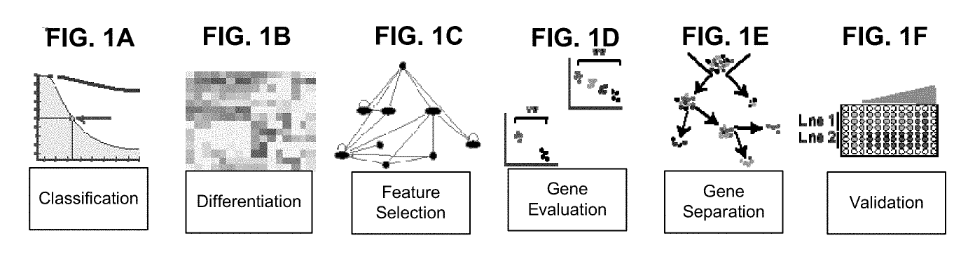

An overview of the workflow used to identify biomarkers associated with drug response is illustrated in FIGS. 1A-1F. For example, processing circuitry in communication with the GDSC via a communication network may select cell lines (e.g., B-cell lines) stored in the GDSC that show response and cell lines that show strong resistance to a drug (e.g., dasatinib) in a “classification” step shown in the workflow step illustrated in FIG. 1A. The processing circuitry then may carry out differential expression, such as by using sig.genes with a False Discovery Rate (FDR) of <0.10 to analyze the cell lines in a “differentiation” step shown in the workflow step illustrated in FIG. 1B. Next, in a “feature selection” step shown in the workflow step illustrated in FIG. 1C, the processing circuitry may analyze gene lists and fold change expression using Ingenuity Pathway Analysis, a web-based analytical tool for ‘omics (e.g., genomics, proteomics, metabolomics, etc.) data. The processing circuitry then may perform Mann-Whitney tests on cell lines determined to be extreme Responders to the drug or extreme Non-Responders to the drug in a “gene evaluation” step shown in the workflow step illustrated in FIG. 1D, which may include ANOVA analysis to determine gene expression trends across the extreme Responders and the extreme Non-Responders. In the workflow step illustrated in FIG. 1E, the processing circuitry may identify statistically significant genes that define a gene signature, and used the gene signature to separate cell lines by response. In the workflow step illustrated in FIG. 1F, the processing circuitry then may evaluate data pertaining to untested cell lines and clinical samples in order to validate the gene signature. In some examples, the validated gene signature produced by the workflow illustrated in FIGS. 1A-1F may enable processing circuitry of a system to determine whether a particular drug is likely to have a therapeutic effect on a particular cell line (e.g., a cell line derived from patient tissue), which may enable a clinician avoid administering one or more drugs that are not likely to have a therapeutic effect on the cell line.

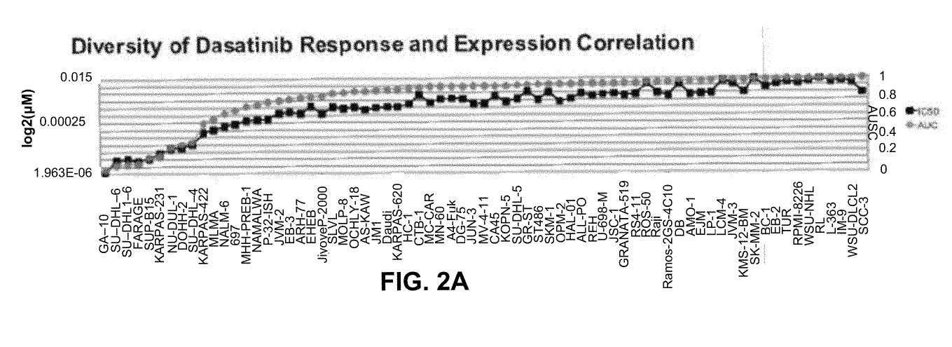

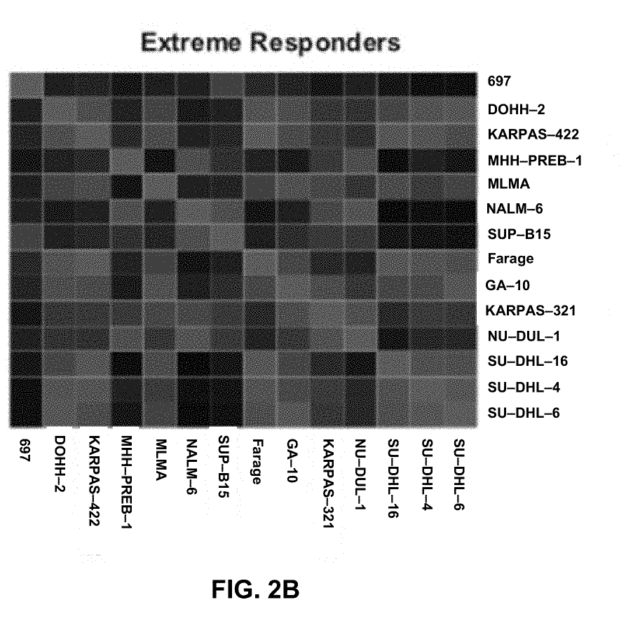

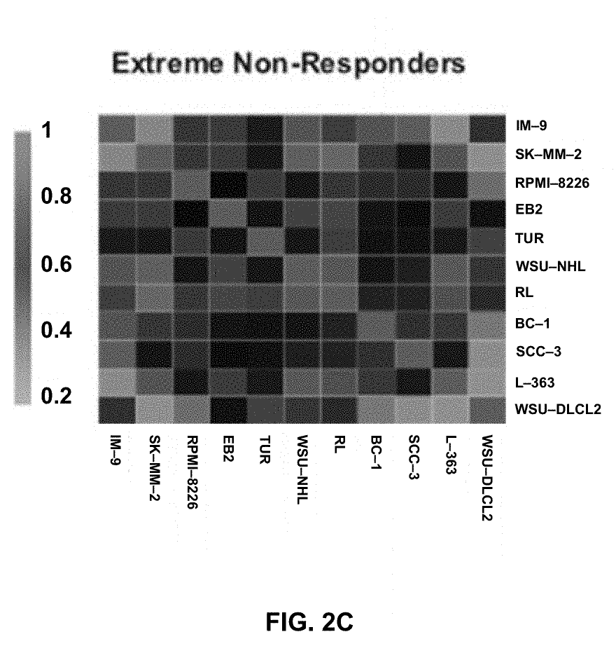

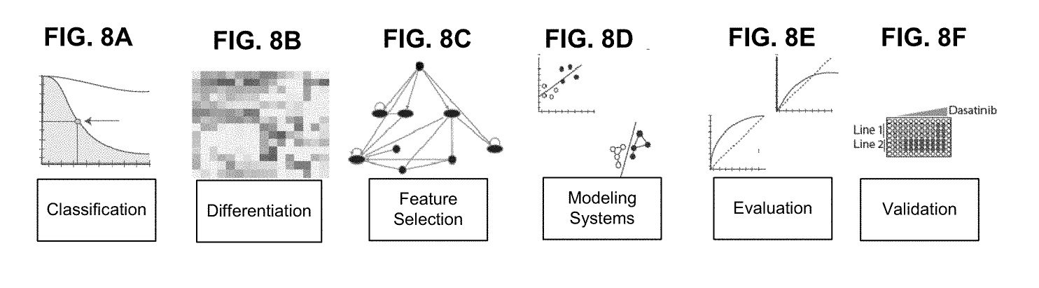

Further aspects of the disclosure will now be discussed, including further details of the techniques described herein, in the context of a working example technique identifying the BCR pathway as an important biomarker that may be used in in predicting cell response to dasatinib. In the context of the working example, FIGS. 2A-7 and FIGS. 9-17 illustrate an application of the workflow of FIGS. 1A-1F and another example workflow used to identify biomarkers of drug response (described below with respect to FIGS. 8A-8F) to the working example technique. The example laboratory techniques described herein for accomplishing routine laboratory tasks, such as the collection of blood and the isolation of serum from blood, as well as others, are not intended to be limiting and may be performed by any suitable laboratory techniques. In addition to the techniques described above, supplementary techniques, as described below, may be employed.

Methods

Expression Analysis

Robust-Multi-Array Average (RMA) normalized expression data and drug response data was downloaded directly from the GDSC website (www.cancerrx.org). CCLE Robust-Multi-Array Average (RMA) normalized expression data was downloaded from Genomicscape.com. Multiple ENSEMBL IDs mapping to the same gene region were averaged. CCLE values were normalized to the GDSC using analysis of 48 cell lines in common between the two data sets. R scripts were used to analyze data as described herein. For example, each R script includes commands that can be entered into the command line of the R statistical computing programming environment. The commands in each R script may call one or more statistical and graphical techniques such as linear and nonlinear modeling, statistical tests, classifications, and other statistical methods. Although R is described here, the data described herein may be analyzed with other programming languages or statistical computing packages in other examples.

Statistical Analysis

Strong Responders' expression values and the highly-resistant Non-Responders' expression values for each gene in the signature were compared using the unpaired, nonparametric, Mann-Whitney test in GraphPad (Prism). Ordinary one-way ANOVA was performed in GraphPad (Prism) on all genes of the gene signature using 4 groupings based on response values: Responders (AUSC<0.85), Partial Responders (AUSC 0.85-0.95), Limited Responders (AUSC 0.95-0.98), and Non-Responders (AUSC>0.98). The Significant Analysis of Microarray (sam) function of the siggenes R package was used in differential expression analysis. The cor( ) function of the R stats package was used to find Pearson correlation coefficients using all expression values between cell lines. All heatmaps, including the Pearson correlation coefficients were rendered using pheatmap.

Tissue Culture

All cell lines were maintained in RPMI-1640 (Lonza) supplemented with 10% FBS (Invitrogen Life Technology), 1× Antibiotic/Antimycotic (Gibco), 1× L-Glutamine (Gibco), and 1 ng/mL of human IL-6. Incubators were humidified and maintained at 37° Centigrade with a 5% CO₂ content.

Dose-Response Assay

Cells were plated at a concentration of 5×105/mL on day 0. On day 1, a dilution series of concentrations (2-fold dilutions from the max dose of 5.12 μM) as well as a DMSO vehicle control were administered in triplicate. On day 4 (72 hours after treatment), cell viability was measured by Cell Titer-Glo Luminescent cell viability assay according to manufacturer's instructions (Promega) and luminescence was read and recorded using Synergy 2 Microplate Reader (Biotek). Maximum viability, assigned as 100% for IC50 or 1 for AUSC calculations, was normalized to untreated controls. IC50 values were estimated by calculating the nonlinear regression using the inhibitor-normalized response equation (variable slope) in GraphPad (Prism).

As done in the calculation of AUSC in the GDSC, wells containing media, drug, but no cells were used to calculate the value for normalizing maximum response, which corresponds to a 0 value. AUSC used the concentrations that overlapped with GDSC doses and substituted the first lowest concentration within the GDSC dilution series for the lower 2 doses tested in lab. AUSC were calculated using GraphPad (Prism).

Results

Classification of Response and Resistance

FIG. 2A represents the diversity of dasatinib response and expression correlation and is described herein with respect to the working example. As shown in FIG. 2A, the 71 B-cell lines were arranged by response, using both Area Under the Survival Curve (AUSC) and IC50. In FIG. 2A, the 71 B-cell malignancies found in the GDSC with dasatinib data are plotted along the x-axis in increasing order of AUSC value. Both log2 (IC50) values (darker boxes, scaled on the primary axis) and AUSC scores (lighter circles, scaled on secondary axis) are represented. The distribution of response favored non-response, with only 14 lines showing a strong response to low doses, and 11 lines showing essentially no response. Specifically, Responders were classified as lines that show an AUSC<0.75, and an IC50 value<maximum drug concentration divided by 4 and Non-Responders were defined as having an AUSC>0.98 and an IC50>maximum dose tested. This resulted in the 14 strong Responder lines versus 11 highly resistant (Non-Responder) lines, representing the extreme ends of response and resistance. The rationale for this classification was that underlying this wide separation of response may be a common expression signature or pathway(s) that can serve as a predictive biomarker.

Pearson correlation coefficient analysis was conducted on all 71 cell lines (see methods). Pearson correlation coefficients were calculated using all available 17,419 gene expression observations within the GDSC. Perfect correlation coefficients of 1 extend along the center diagonal from upper left to lower right as each cell line is compared to itself. The lowest correlation coefficients are found between Non-Responders, which may be indicative of their expression diversity. Notably, Responders shared more similarity than Non-Responders (FIGS. 2B, 2C). What is apparent is the Non-Responders, as a group, are far more diverse in gene expression than those cells that are within the Responder group. Thus, further attention was directed to the features that define the Responders that are distinct from the gene expression of the diverse Non-Responders.

Differential Gene Expression and Feature Selection

Differential gene expression between the Responder and Non-Responder lines was performed using Significance Analysis of Microarray (see Methods), with a False Discovery Rate limited to 10%. This resulted in 228 genes to further analyze for their relevance to the dasatinib response.

Ingenuity Pathway Analysis (IPA) (Qiagen) uses an extensive, curated literature bank to identify molecule interaction and pathway regulation. Data is uploaded and populates the knowledge base interactions. Significant p-values are generated when genes fall within a pathway in a non-random manner. Further, pathway activation scoring is achieved by similarly populating relevant pathways and the directionality of the gene expression. Analysis of major canonical pathways provides both the p-value (likelihood of true positive) and z-score (directional impact of activation/de-activation).

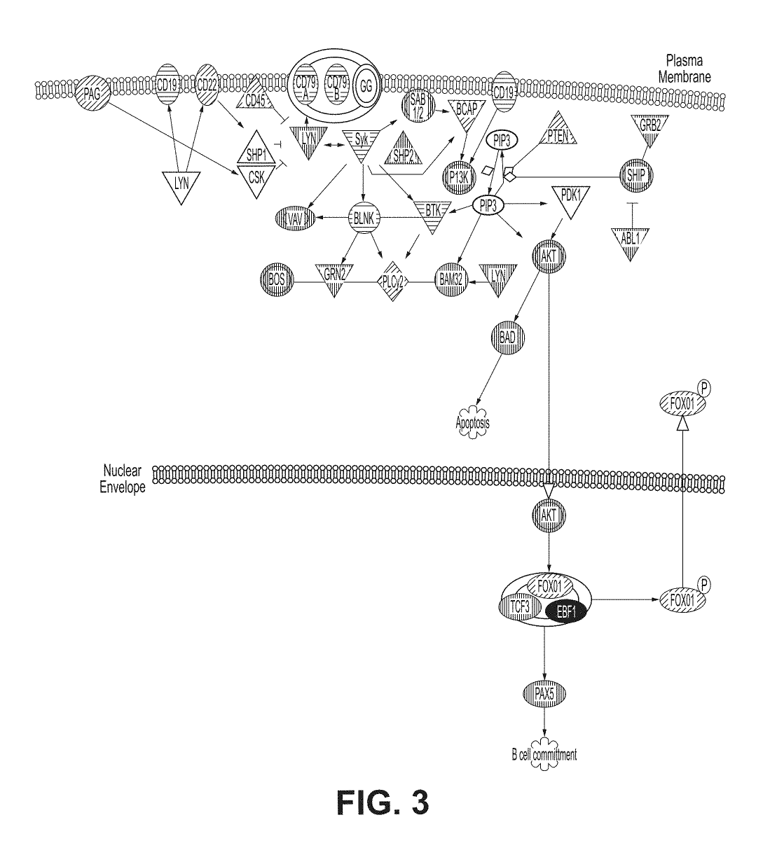

FIG. 3 illustrates the activation of the BCR pathway in Extreme Responders. Genes of the BCR pathway represented as expression ratios of highly sensitive Responders relative to Non-Responders. The different fill patterns within the icons represent different fold-changes between groups. White fill (e.g., as shown for PDK1) indicates the lowest fold-change. Vertical lines (e.g., as shown for GRB2) indicate the second-lowest fold-change. Gray fill (e.g., as shown for ABL1) indicates the third-lowest fold-change. Diagonal lines (e.g., as shown for PTEN) indicate the third-highest fold-change. Horizontal lines (e.g., as shown for CD19) indicate a second-highest fold-change. Black fill (e.g., EBF1) indicates the highest fold-change. Pathway analysis was conducted using IPA on the 228 differentially expressed genes and their log-fold ratio data of Responders relative to Non-Responders. Notably, the top canonical pathway with a significant z-score (z-score=2, which is 2 standard deviations from the mean) was the B-cell-receptor (BCR) pathway (p-value=0.013). Using the log-fold ratio scores of Responders relative to Non-Responders, molecules of the BCR pathway reflected a uniquely activated pattern in the Responders. In such a manner, processing circuitry of a system may enable the system to identify a gene signature associated with the pattern analysis, which may help enable a clinician to administer a drug having an efficacy associated with the gene signature.

BCR and B-Cell Development

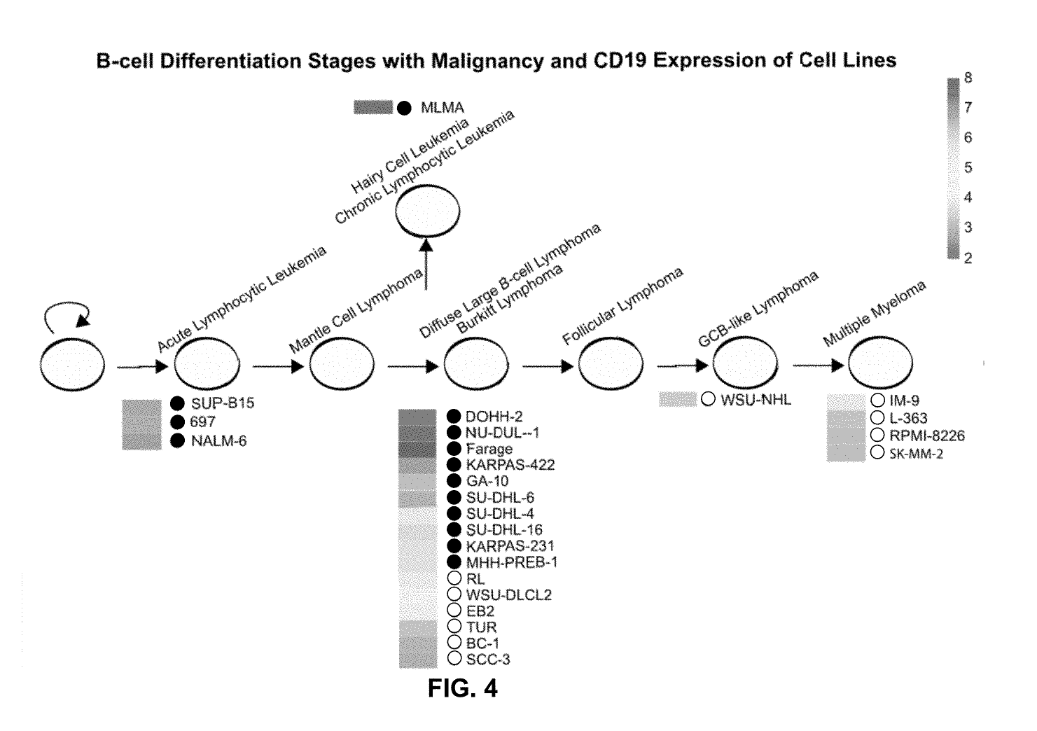

FIG. 4 illustrates B-cell differentiation stages with malignancy and CD19 expression of cell lines. In FIG. 4, the maturation of B-cells is represented from left to right. Malignancies arising from corresponding stages of B-cell development are depicted along with cell lines representing those malignancies in the same vertical axis. Next to cell line names are either a black dot (Responders) or a white dot (Non-Responders) along with their corresponding log2 fluorescence intensity of CD19, the marker found most potent for prediction of dasatinib response. The BCR pathway has been demonstrated to be active at different stages of B-cell development. As shown in FIG. 4, the activation of various oncogenes and tumor suppressor genes gives rise to the malignancies at different stages of B-cell development. The distribution of response to dasatinib along the B-cell differentiation path indicates cancers arising from a pre-B-cell or an early B-cell may be more likely to respond than B-cells or plasma cells later in development that notably have decreased expression of BCR genes. In some examples, this technique may be applied to pathways other than the BCR pathway to identify expression levels associated with different stages of development of a cell line (e.g., a B-cell line or other type of cell line), such as to identify expression levels of genes of a gene signature that may be associated with the development of one or more conditions other than myeloma. This technique also may be applied to identify response or resistance of such cell lines to drugs other than dasatanib at different stages of development of the cell line, or to identify other attributes of such cell lines at different stages of cell development that may have clinical or other significance.

Evaluation of the Genes in the BCR Pathway

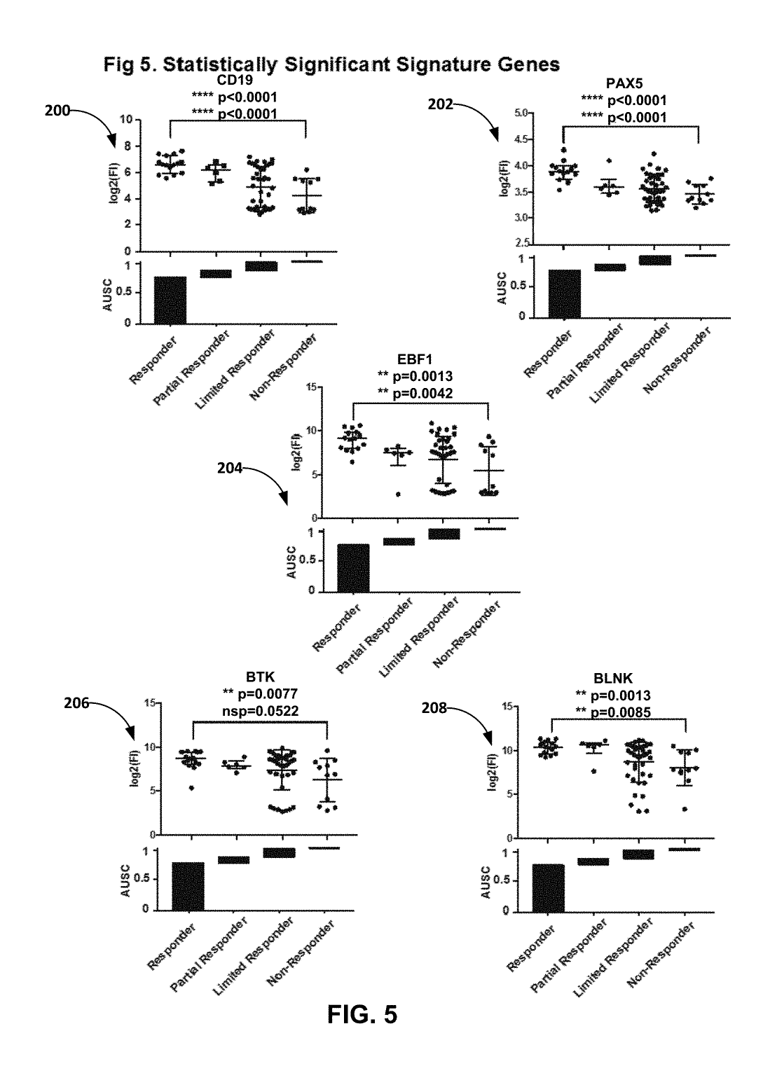

FIG. 5 illustrates statistically significant signature genes. The differentially expressed genes of the BCR pathway were further characterized between the Responders (high expression) and Non-Responders (low expression). Mann-Whitney tests of individual genes of the BCR pathway were performed, and the expression value of 5 genes with the most significant associations are shown across the 71 cell lines in the GDSC collection (FIG. 5): CD19 (graph 200), PAX5 (graph 202), EBF1 (graph 204), BTK (graph 206), and BLNK (graph 208). As shown in FIG. 5, each gene in the 5-gene signature is represented with cell lines binned according to AUSC and their log2 expression values. The categorical responses depicted in each graph of FIG. 5 correspond to AUSC values as follows: Responder (0-0.75), partial Responder (0.75-0.85), limited Responder (0.85-0.98), and Non-Responder (>0.98). Mann-Whitney tests p values assessing the Responders versus Non-Responders are listed above the p values for ANOVA across all categories.

Intermediate response groups were included to identify an expression trend across the full range of responses. These groupings included cell lines with a partial response (AUSC between 0.85-0.95) and limited response (AUSC between 0.95-0.98). Significant p-values between adjacent grouping were not observed. However, ANOVAs were performed using all 4 groupings and indicated 4 of the 5 genes showed highly significant differences between the Responders and Non-Responders (ANOVA p-values listed underneath Mann-Whitney test p-values in FIG. 5), and trends of decreasing expression across the increasing resistance groupings.

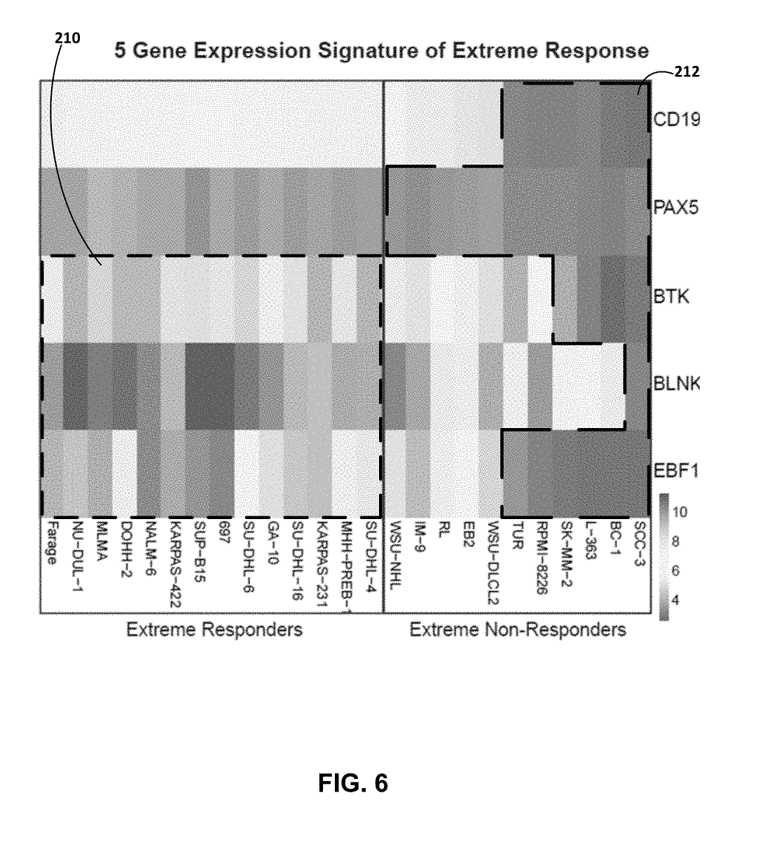

FIG. 6 illustrates the gene expression signature of extreme response. The expression of the 5 genes is shown as a heatmap comparing the extreme Responders and the extreme Non-Responders. Notably, CD19 alone showed a significant association with response (p<0.0001). Unsupervised clustering of the gene expression signature that discriminates Responders from Non-Responders Cell lines are listed along the x-axis while the 5 genes most associated with dasatinib response are on the y-axis. Expression values are log2 transformed fluorescence intensities. The 14 extreme Responders are grouped in box 210 on the left of the heatmap, the 11 extreme Non-Responders are grouped in box 212 on the right. The dynamic ranges of each gene in the signature is not always reflective of its contribution to identifying response as can be seen in the case of PAX5. This gene is highly significant in differentiating response, although its absolute values vary subtly, but significantly (see FIG. 5, p<0.0001).

Validation

Independent Cell Lines

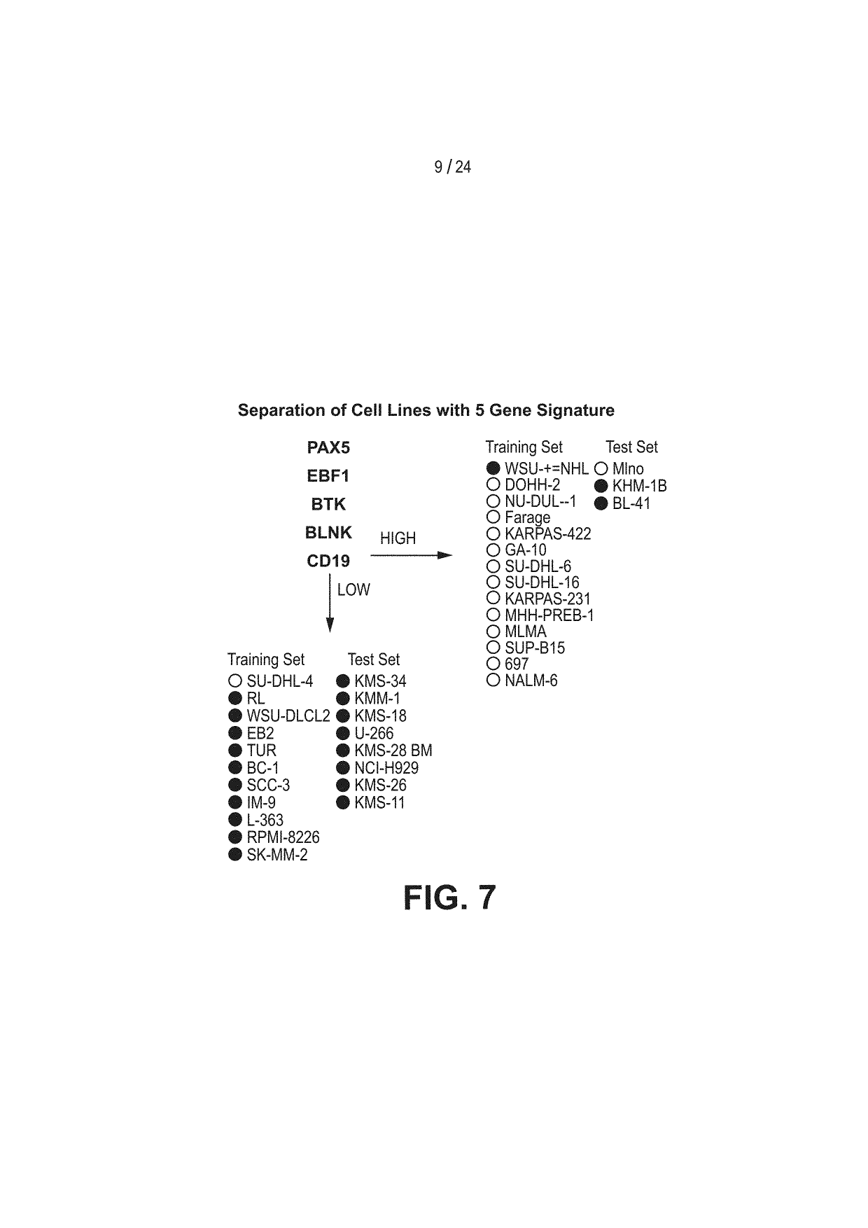

FIG. 7 illustrates the validation of cell lines with the 5-gene signature. Eleven cell lines not included in the modeling had available gene expression data (8 from CCLE, 3 from GDSC). These were tested in-house for dasatinib response. Training and Test Lines were evaluated based on their expression values relative to the extreme responding lines. Lines with expression values less that the average of the Responders and Non-Responders of the training set are binned into Responder and Non-Responder. The test set of 11 cell lines had 1 true positive, 2 false positives, 8 true negatives, and no false negatives. The CCLE/GDSC expression platforms were then normalized to one another to obtain comparable values (not shown). The binary classification of these lines based on the 5-gene signature into Responder (AUSC<0.85) and Non-Responder (AUSC>0.85) demonstrated accurate prediction in 9 of the 11 lines (FIG. 7). In analysis of intermediate responding lines, 100% of lines (n=20) that had low CD19 expression also had AUSC scores of >0.85. These 20 cell lines were not in the response modeling set.

Clinical Associations

MM lines (plasma cells) rarely express CD19, and show a low activation of the BCR pathway. A recent clinical study (NCT00429949) of relapsed, refractory, or plateau phase MM patients was discontinued after using dasatinib as a single agent and observing a partial response in only 1 of 21 enrolled in the study.

Waldenstrom's macroglobulinemia (WM) is also a plasma cell malignancy, but in contrast to multiple myeloma is CD19+, and has recently been described to express an activated BCR pathway. Based on the 5-gene signature, it was predicted that these malignancies would respond to dasatinib and thus would represent an independent validation set of Responders. Indeed, WM primary patient lines exhibited good response to dasatinib in primary patient samples (n=32) supporting the findings that the expression of CD19 and 4 other molecules of the BCR pathway are consistent in a dasatinib response.

Discussion

Dasatinib response of B-cell malignancies is described herein as corresponding to cell lines falling into two extreme groups: Responders and Non-Responders. From this binary categorization, comparisons were made between the groups. Differential expression revealed a set of genes, that, when uploaded to IPA, was most significant for the BCR pathway. The Responders had an activated pathway and conversely, the Non-Responders had a de-activated BCR pathway. Five genes of this pathway; i.e., CD19, EBF1, BTK, BLNK, and PAX5, were used to sort the 25 original cell lines into their Responder/Non-Responder groupings.

Eleven independent cell lines were likewise binned according to their expression values for each of the five genes. Nine of the 11 were appropriately categorized. WM patient primary samples have expression patterns indicative of response. As a malignancy, WM primary cell lines are responsive to dasatinib. When examined in total, of the 98 cell lines and clinical samples examined, 94 responded according to the prediction of the 5 genes in the BCR pathway.

Further supporting the findings here is work done in mantle cell lymphoma (MCL). MCL is mid-stage B-cell malignancy and may or may not express CD19. Two MCL lines were treated with low doses of Bortezomib over time to better understand mechanisms of resistance in MCL. This acquired bortezomib resistance (BTZ-R) was accompanied by a re-activation of the BCR pathway (i.e., these cells had increased expression and phosphorylation of BCR components above parental lines). Notably, along with this re-activation, a collateral increase in sensitivity to dasatinib was observed. The BTZ-R lines responded to doses of dasatinib 10-fold less concentrated than parental lines. These observations held true in mouse xenografts of the MCL line pairs treated with dasatinib as well. These data further support the finding that CD19 and the BCR pathway are consistent biomarkers in B-cell lineage cells response to dasatinib.

As described herein, cell line expression and drug response can be interrogated through differential expression and pathway analysis to find meaningful relationships and identify biomarkers of drug response. The associations identified in the modeling set provided a robust association with actual clinical outcomes. In addition, the patient correlations remained significant to predict response across the B-cell developmental pathway. The working example described herein is just one example of the use of available data bases to develop response signatures. Similar approaches may provide gene signatures across many other drugs within the GDSC, CCLE, or similar large cell line data bases. Ultimately, expression signatures may add important biomarkers that better direct therapeutic approaches to the treatment of other diseases and conditions, and/or may identify attributes of cell development having other clinical, scientific, aesthetic, or other significance.

Additional aspects of the process described above for using databases to develop predictive biomarkers of drug response in hematologic malignancies are illustrated in FIGS. 8A-13.

FIGS. 8A-8F illustrate an example of another workflow used to identify biomarkers associated with drug response. In FIG. 8A, cell lines representing extremes of drug response were chosen from all available B-cell malignancies. In FIG. 8B, differential expression using sig.genes with an FDR of <0.10 was used to describe cell lines. In FIG. 8C, gene lists and fold change expression were analyzed using Ingenuity Pathway Analysis. In FIG. 8D, Generalized Linear Regression and k-Nearest Neighbor (using the caret package) was used to create models. In FIG. 8E, Receiver Operator Curves were used to describe performance and compare models. In FIG. 8F, untested lines were used to test (e.g., validate) the prediction algorithm.

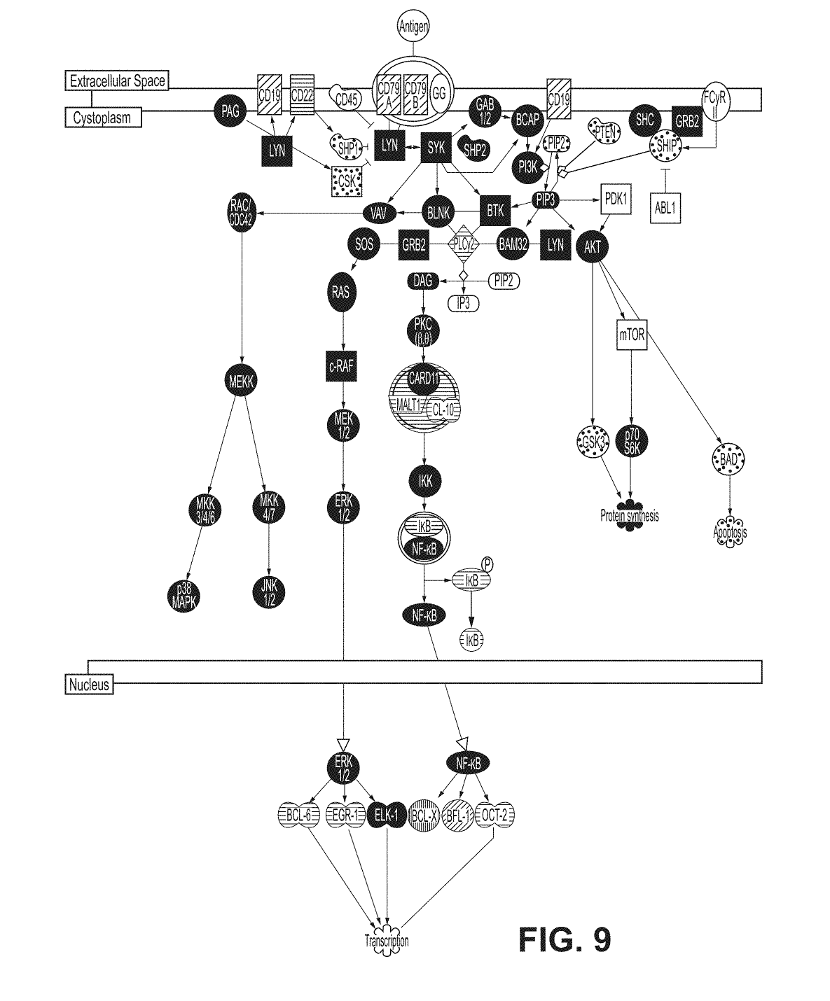

FIG. 9 illustrates the activated B-cell receptor signaling pathway (BCR), which includes 90 genes. Initial observations of differential expression revealed several key genes involved in the canonical B-cell Receptor Signaling (BCR) pathway upregulated in cell lines with response to dasatinib at nM concentrations. FIG. 9 shows an activated pathway in which differential expression of gene products in the BCR pathway is identified by different fill patterns within the icons. As shown in FIG. 9, expression of nearly all products in the BCR pathway was found to be up-regulated in cell lines with response to dasatinib at nM concentrations (i.e., the Responder cells). Among the up-regulated products in the BCR pathway, the least up-regulated products are indicated by vertical lines (e.g., BCL-X). The second-least up-regulated products are indicated by closely-spaced diagonal lines (e.g., BFL-1). Moderately up-regulated products are indicated by horizontal lines (e.g., EGC-1). The second-most up-regulated products are indicated by black fill (e.g., ELK-1). The most up-regulated products are indicated by wide-spaced diagonal lines (e.g., CD-19). The expression of seven products was found to be down-regulated: SHP1, CSK, PIP2, PTEN, SHIP, GSK3, and BAD (indicated by dotted fill). The expression of four products was found not to be differentially expressed: CD45, IGG, FCYRII, and IP3 (indicated by white fill).

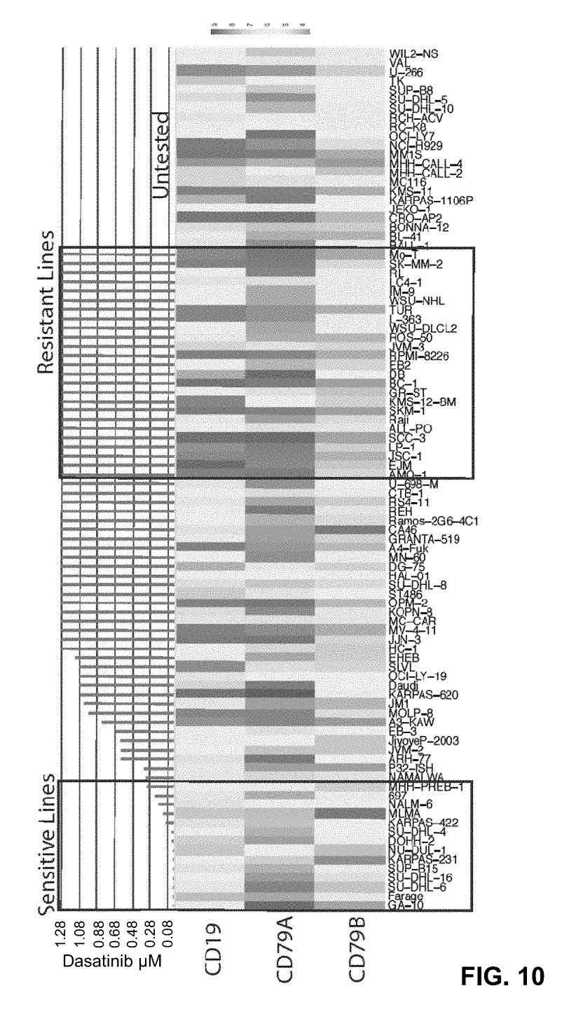

FIG. 10 illustrates an expression comparison of three BCR components in cell lines in increasing order of dasatinib concentration needed to induce 50% reduction in metabolism. Two of the significantly differentially expressed genes are part of the B-cell Receptor Signaling canonical pathway. Kim et. al (2015) demonstrated these same molecules, as well as another component of the BCR complex, to correspond with an acquired sensitivity to dasatinib. As shown in FIG. 10, relative over-expression of CD19, CD79A, and CD79B is enriched in sensitive lines.

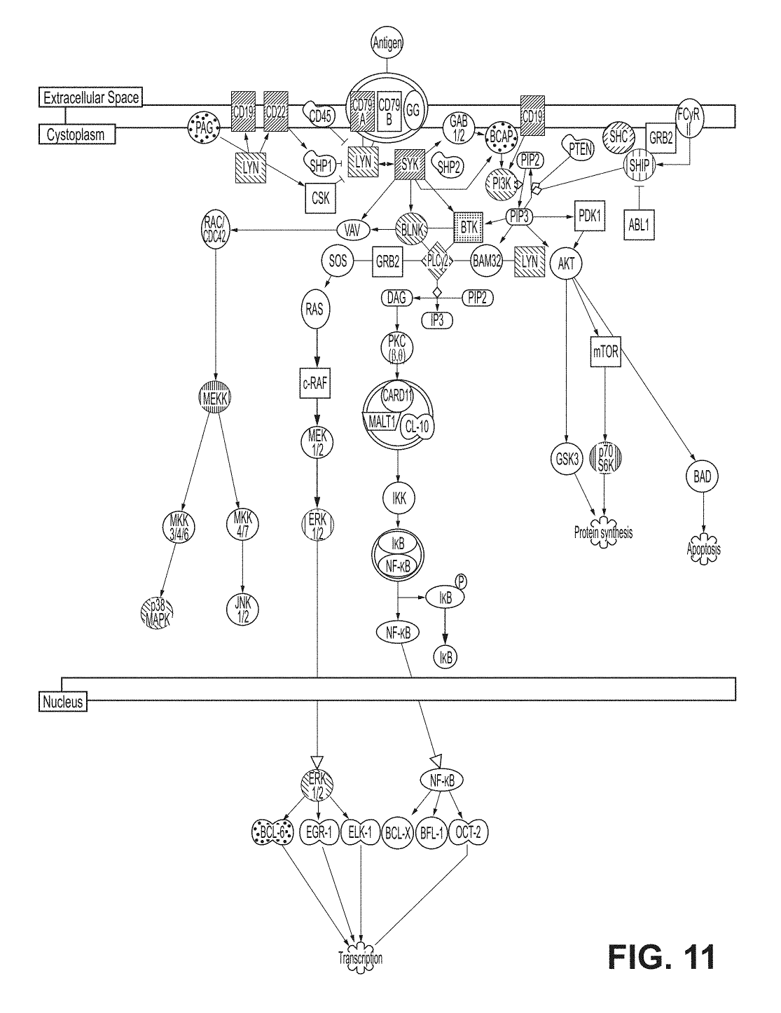

FIG. 11 illustrates an Ingenuity Pathway Analysis of the BCR pathway of Non-Responder cells. As with FIG. 9, differential expression of gene products in the BCR pathway shown in FIG. 11 is identified by different fill patterns within the icons. As shown in FIG. 11, the Non-Responder cells relatively down-regulate components of the BCR signaling pathway. Expression of PAG LYN, CD19, CD22, CD79A, Syk, BLNK, BTK, PLCy2, BCAP, PI3K, SHIP, p38 MAPK, and ERK1/2 was found to be down-regulated. Among the down-regulated products in the BCR pathway, the least down-regulated products are indicated by widely-spaced vertical lines (e.g., SHIP). The second-least down-regulated products are indicated by diagonal lines running from top left to lower right (e.g., PI3K). Moderately down-regulated products are indicated by cross-hatching (e.g., BTK). The second-most down-regulated products are indicated by closely-spaced diagonal lines running from lower left to top right (e.g., CD19). The most down-regulated products are indicated by dotted fill (e.gp. BCAP). Expression of MEKK, SHC, and p70 S6K was found to be up-regulated, with SHC more up-regulated (indicated by widely-spaced diagonal lines running from lower left to upper right) than MEKK and p70 S6K (both indicated by closely-spaced vertical lines). Relative down-regulation corresponds with extreme resistance to dasatinib.

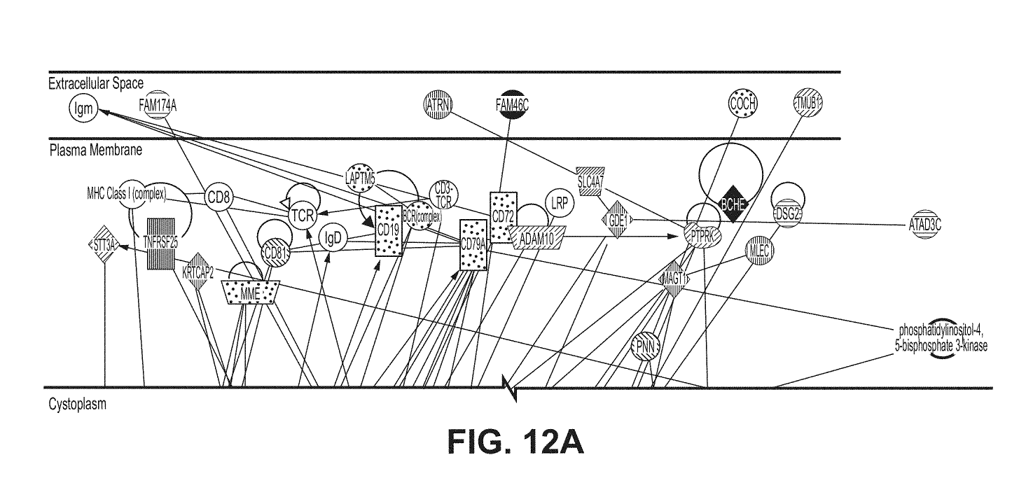

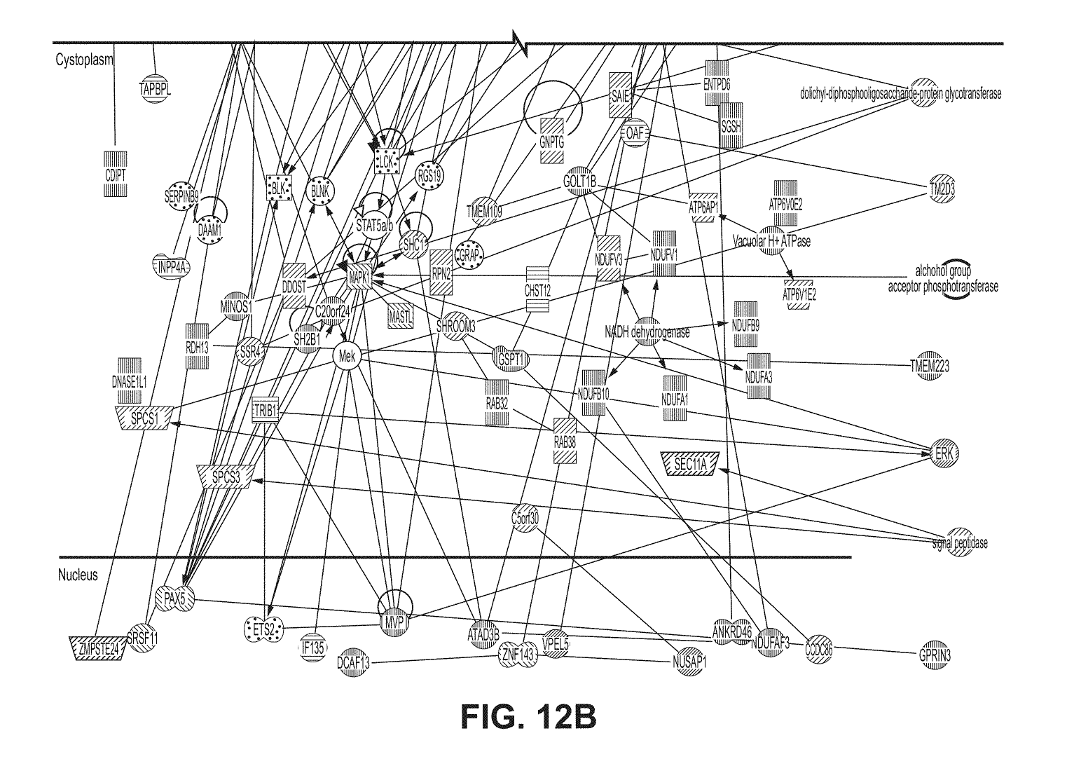

Together, FIGS. 12A and 12B illustrate the genes of the BRC signaling pathway networked within 2-degree relationships with SRC molecules to create a potent prediction list. For the sake of clarity, the BRC signaling pathway is illustrated in two portions. FIG. 12A illustrates the portion of the BRC signaling pathway that may be located within the extracellular space or within the plasma membrane. FIG. 12B illustrates the portion of the BRC signaling pathway that may be located within the cytoplasm or within the nucleus. Networked relationships between expression products that may be located within the plasma membrane and expression products that may be located within the cytoplasm may be observed by joining FIGS. 12A and 12B along the line between the plasma membrane space and the cytoplasm space. As with FIGS. 9 and 11, differential expression of gene products in the BCR pathway shown in FIGS. 12A and 12B is identified by different fill patterns within the icons. Among the down-regulated products in the BCR pathway, the less down-regulated products are indicated by diagonal lines running from top left to lower right (e.g., PNN in FIG. 12A and PAX5 in FIG. 12B). The more down-regulated products are indicated by dotted fill (e.g., CD19 in FIG. 12A and SERPINB9 in FIG. 12B). Among the up-regulated products in the BCR pathway, the least up-regulated products are indicated by vertical lines (e.g., MLEC in FIG. 12A and SGSH in FIG. 12B). The second-least up-regulated products are indicated by diagonal lines running from lower left to upper right (e.g., PTPRK in FIG. 12A and GNPTG in FIG. 12B. The second-most up-regulated products are indicated by horizontal lines (e.g., DSG2 in FIG. 12A and OAF in FIG. 12B). The most up-regulated products are indicated by black fill (e.g., FAM46C in FIG. 12A).

To generate the networked BRC signaling pathway illustrated in FIGS. 12A and 12B, differentially expressed genes were uploaded to Ingenuity Pathway Analysis. Networks were queried to include SRC molecules. All networks were merged and genes more than two relationships from differentially expressed genes were removed. The network analysis list involves 469 genes.

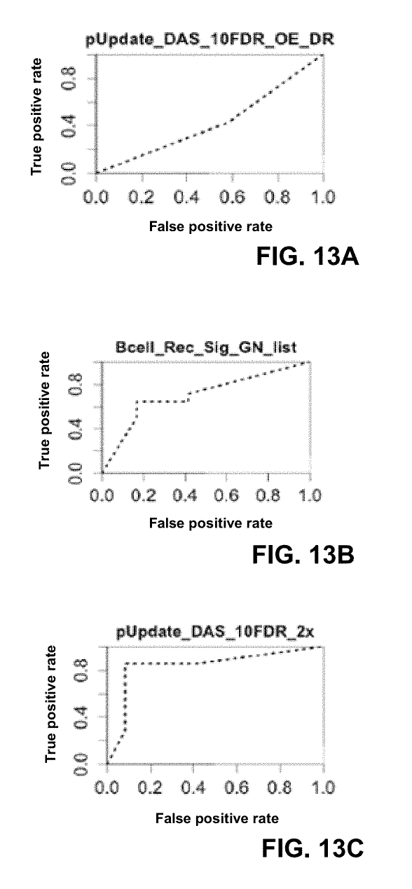

FIGS. 13A-13C illustrate that Receiver Operator Curves produced using a “leave-one-out” strategy to assess model performance indicate pathway curation increases predictive power. As shown in FIG. 13A, using only differentially expressed genes creates a model with less-than-chance efficacy. As shown in FIG. 13B, using all canonical B-cell Receptor Signaling pathway genes greatly improved performance. As shown in FIG. 13C, all genes networked with differentially expressed SRC molecules provided the best model. Differential expression between responder and non-responder cell lines provides us with a starting point by which to describe expression signatures predictive of drug response. Here, by curating genes differentially expressed, predictive power can be extended. Databases such as the GDSC and the Cancer Cell Line Encyclopedia provide a useful tool in prediction of drug response in the clinic. However, the cell lines of these collections, though large, describe a limited view of perturbed cancer pathways in the human population. Thus, including molecules from network analysis in modeling may better predict response to dasatinib or other drugs.

The examples described above represent the use of available data bases to develop response signatures with respect to the drug dasatinib. Similar approaches may provide gene signatures across many other drugs within the GDSC, CCLE, or similar large cell line data bases. Ultimately, expression signatures may add important biomarkers that better direct therapeutic approaches.

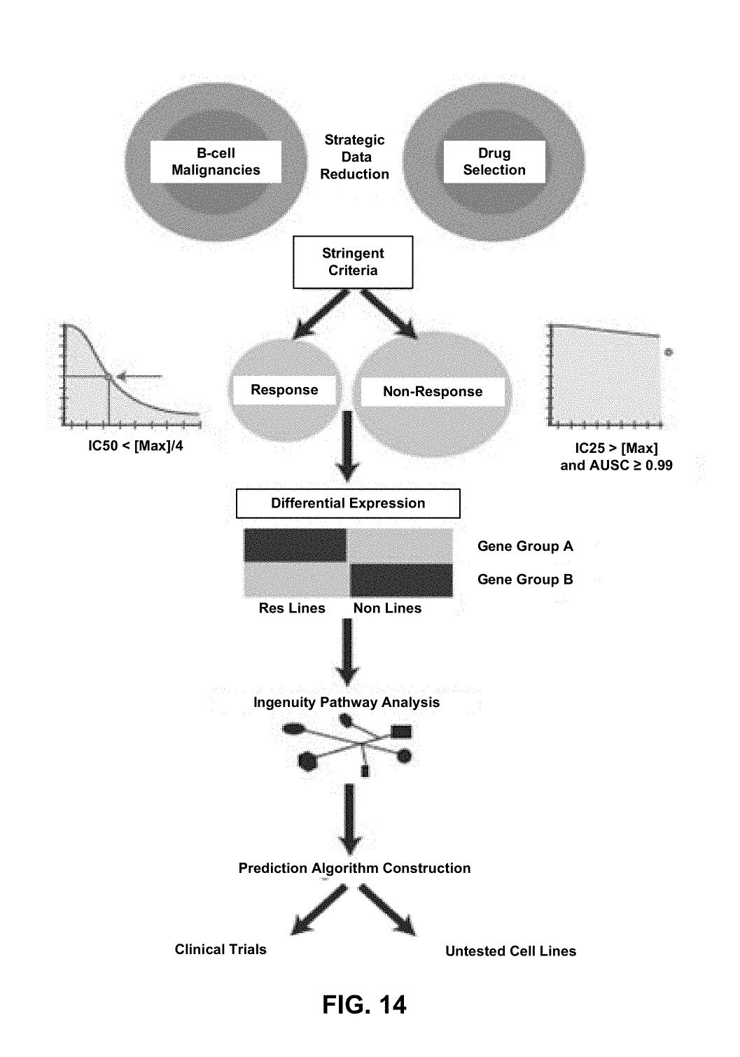

Another example of the use of available data bases to develop response signatures to the drug WH-4-023 is illustrated in FIGS. 14-18. Response profiles of over 50 human myeloma cell lines to therapeutic drugs used in the clinic have been determined. A computational pipeline that uses RNAseq data to create gene expression signatures associated with cell line response or resistance is used, and demonstrates successful application of these signatures in predicting therapeutic response in myeloma clinical trials. Further, gene expression profiles of cell lines after specific drug resistance has emerged to identify signatures of response to other drugs, providing more effective therapeutic options.

In some examples, a publicly-available database can be used to expand studies of drug-response expression signatures. The Genomics of Drug Sensitivity in Cancer (GDSC) (http://www.cancerrxgene.org) is an expanding database with a collection of over 1000 cell lines and 140 drugs. The collection includes 71 B-cell malignancies lines characterized by gene expression analysis as well as drug responsiveness. These data can be used to extract gene expression signatures of response and resistance to selected drugs across the GDSC B-cell hematologic malignancy derived cell lines. Initial analyses have identified differential expression signatures of the responder versus the non-responder lines for an expanding number of drugs. These data may be used to inform a prediction algorithm capable of identifying response in cell lines as of yet untreated and patient clinical trials. Thus, a GEP response/resistance signatures to drugs using the GDSC data base B-cell line response/resistance to WH-4023 (a proposed Src/Abl inhibitor) has been developed. IPA network analysis reveals multiple pathways affecting response/resistance to WH-4-023.

Some patients respond to treatment, while others do not. Drug response coupled with gene expression data through publicly-available sources can be used as a tool to determine what probesets are most valuable in determining response or resistance. Using the GDSC data base, differentially expressed drug-response gene signatures can be used to identify profiles of cell lines and tumors that will respond to an individual therapy. Approaches may include: cell lines of similar lineage, strict response criteria, clustering of Non-Responders, inclusive Differential Expression Analysis, Pathway Analyses for Biological Relevance, and Prediction Algorithm using informed input.

FIG. 14 illustrates an example of another workflow used to identify biomarkers associated with drug response.

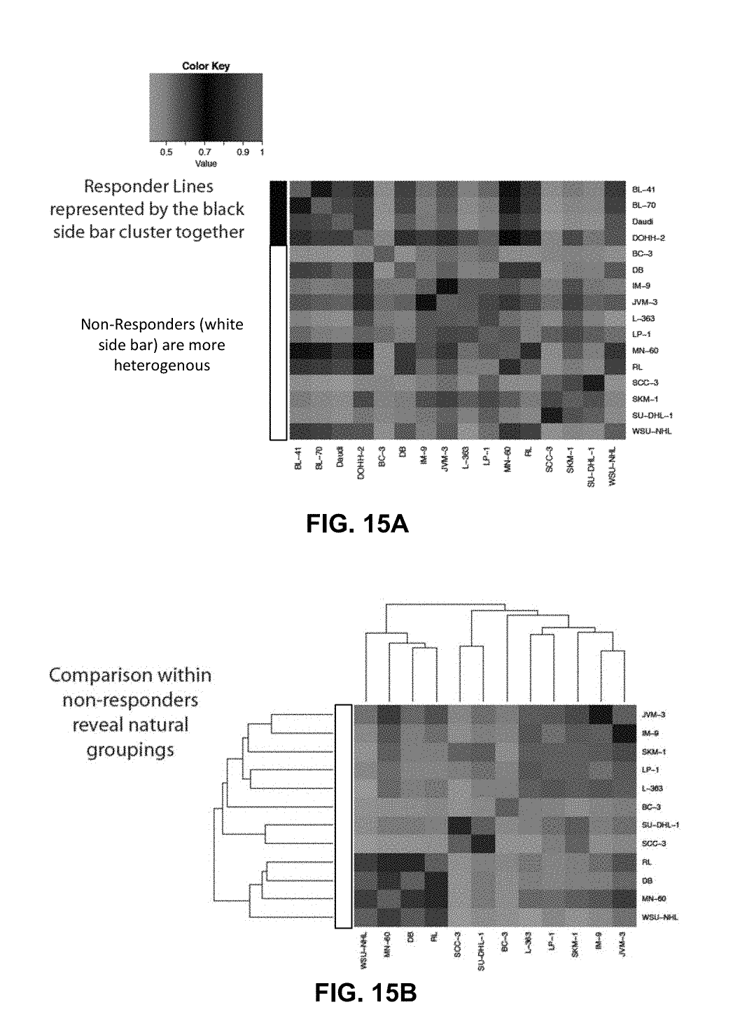

FIG. 15A-15B illustrate pairwise analysis and k-means clustering that shows expression-profile relationships among cell lines. FIG. 15A provides a comparison of the expression profiles of Responder cell lines and Non-Responder cell lines. Perfect correlation coefficients of 1 can be seen along the diagonal as each cell line is compared to itself. The Responder cell lines, designated by the black bar along the y-axes in FIGS. 15A and 15B, display greater similarity of expression profile than the Non-Responder cell lines, designated by the white bar along the y-axes in FIGS. 15A and 15B. However, as shown in FIG. 15B, comparison within the Non-Responders revealed some natural groupings.

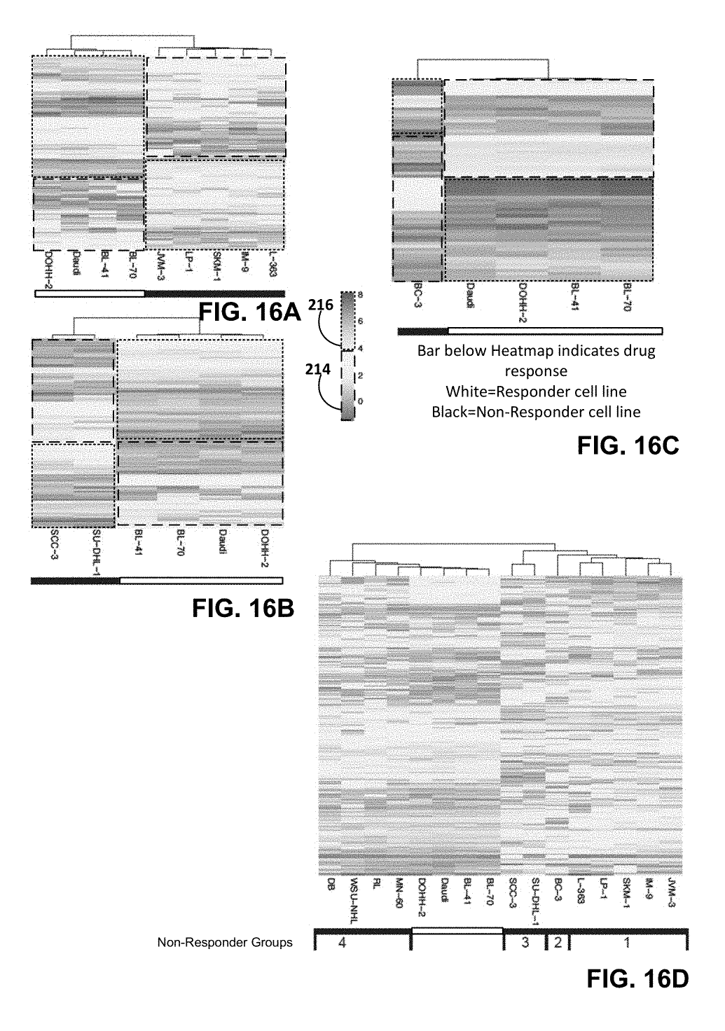

FIG. 16A-16C illustrate differential expression of Responder cell lines (represented by a white bar below an x-axis) compared to three groups of Non-Responder cell lines (represented by a black bar below an x-axis). Probesets (expressed genes) are represented on the y-axes and cell lines on the x-axes. In each of FIGS. 16A-16C, differential expression of the cell lines is shown as a heatmap comparing one or more Responder cell lines to one or more non-Responder cell lines. In each of the heatmaps, groups of genes that are generally expressed at a relatively lower level (i.e., expression values of zero to approximately 4) are outlined with dashed line 214, as shown in the legend positioned between FIGS. 16A-16C. Groups of genes that are generally expressed at a relatively higher level (i.e., expression values of approximately 4 to 8) are outlined with dashed line 216, as shown in the legend positioned between FIGS. 16A-16C. As shown in each of FIGS. 16A-16C, probesets are differentially expressed between Responder cell line groups and Non-Responder cell line groups. Thus, FIGS. 16A-16C illustrate that there consistently is differential expression between Responder cell lines and Non-Responder cell lines, even when different groups of Responder cell lines and Non-Responder cell lines are compared. FIG. 16D illustrates a combined comparison of the three Non-Responder groups to the Responder group, resulting in 427 unique probesets displayed across groups.

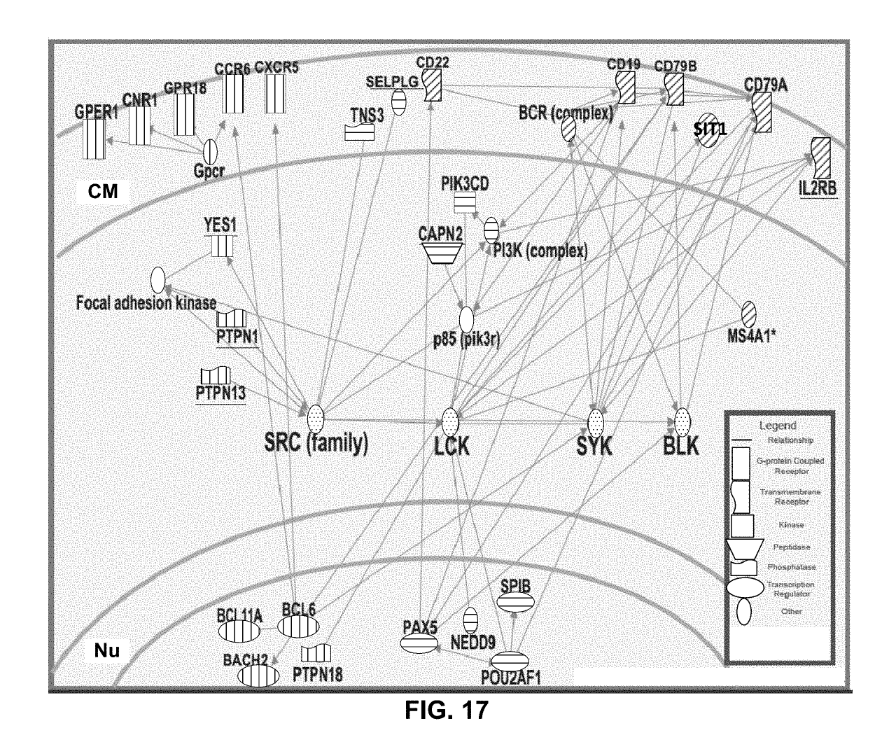

FIG. 17 illustrates individual Non-Responder groups and their resulting networks. The different types of expression products shown in the network of FIG. 17 is illustrated in the Legend box. As shown in FIG. 17, probesets differentially expressed from 3 groups of non-responsive cell lines reveal separate networks that converge on Src kinases. White fill (e.g., as shown for PI3K complex) indicates the lowest fold-change. Horizontal lines (e.g., as shown for PTPN13) indicate the second-lowest fold-change. Vertical lines (e.g., as shown for CAPN2) indicate a fold-change of moderate magnitude. Diagonal lines (e.g., as shown for CD22) indicate a second-highest fold-change. Dotted fill (e.g., LCK) indicates the highest fold-change. This supports the hypothesis that non-responders are heterogenous in the way in which they are resistant to the inhibition of Src. Further, due to the small number of lines in each grouping, networked molecules not identified as differentially expressed should be considered while creating an informed prediction algorithm that will be successful outside of the test set.

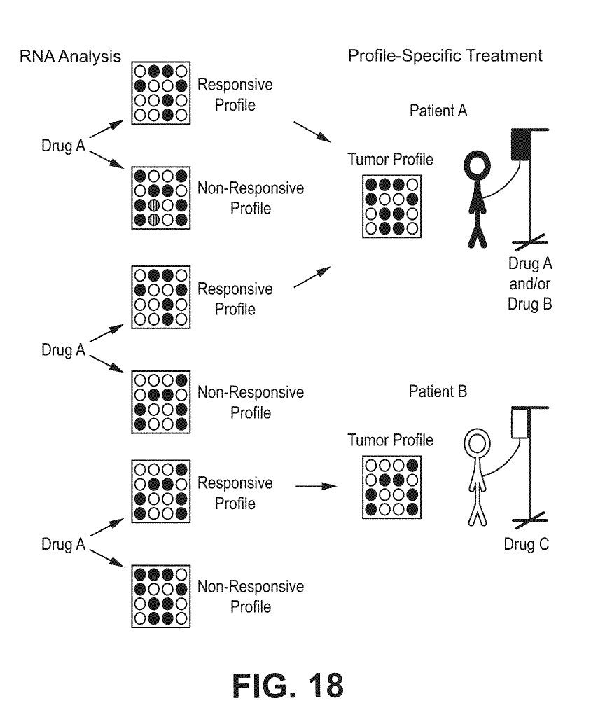

FIGS. 18-21 illustrate example techniques and system components of a system, including processing circuitry, configured to generate a drug-response prediction in response to receiving a gene-expression profile of patient tissue affected by a disease or condition (e.g., tissue from a patient tumor) and a request for the report of the drug-response prediction of the patient tissue. The example techniques and system components illustrated in FIGS. 18-21 may be used to carry out one or more portions of the workflow of FIGS. 1A-1F and the workflow of FIGS. 8A-8F.

FIG. 18 illustrates a method of predicting drug response based on expression signatures. “Responsive” expression profiles for cell lines that respond to a particular drug (e.g., Drug A, Drug B, or Drug C) may be determined, and an expression profile for a cell line derived from a patient's tumor (i.e., a tumor profile) may be determined and compared to the Responsive expression profile for one or more drugs. In the examples illustrated in FIG. 18, differential gene expression in the expression profiles is indicated by the different fill patterns within the circles of the illustrated RNA analysis. Circles having black fill represent up-regulated gene expression, whereas circles having white fill represent down-regulated gene expression. Circles having vertical-line fill represent gene expression that is neither up-regulated nor down-regulated. If a patient's tumor profile is determined to be sufficiently similar to a drug's Responsive expression profile, then the patient may be expected to respond to that drug. For example, as shown in FIG. 18, Patient A may be expected to respond to either Drug A or Drug B, and Patient B may be expected to respond to Drug C, based on the patients' respective tumor profiles. A heme malignancy gene expression profile (GEP) chip that predicts patient response to multiple therapies may be created with this information, giving clinicians and patients direction and confidence with their choice in therapy. Such GEP chips may be used, in other examples, to predict patient response to therapies for conditions other than heme malignancies, such as other cancers or diseases, and/or may be used to identify other aspects of gene expression having other clinical, scientific, aesthetic, or other significance.

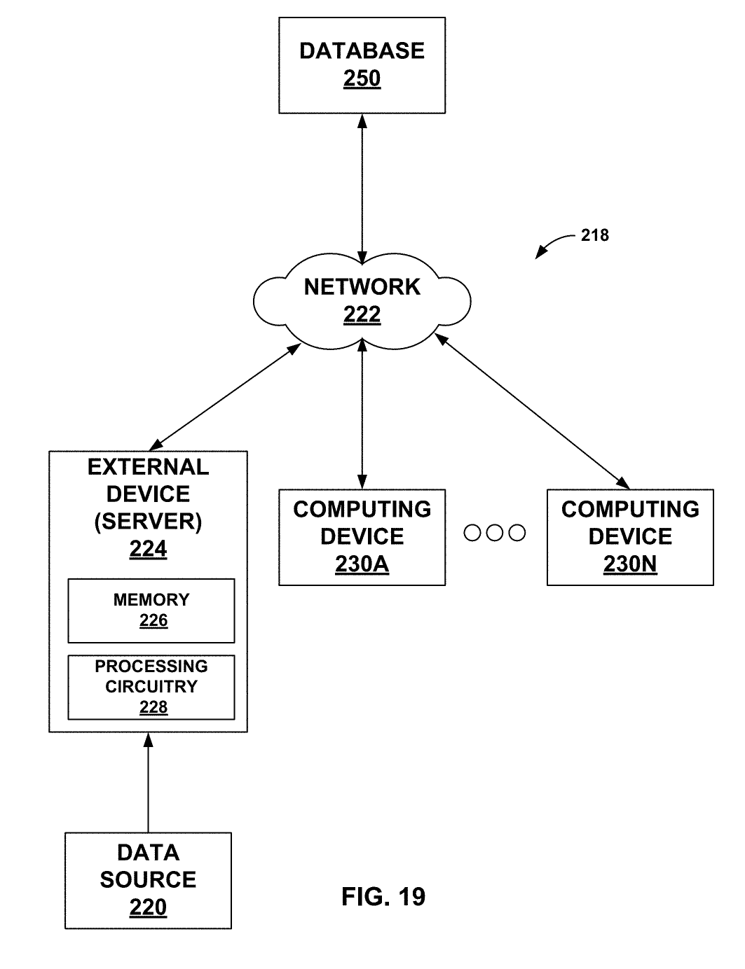

FIG. 19 is a functional block diagrams of an example system 218 configured to perform the techniques described in accordance with the disclosure. As illustrated in FIG. 19, various aspects of the techniques may be implemented within processing circuitry of one or more device, including one or more microprocessors, DSPs, ASICs, FPGAs, or any other equivalent integrated or discrete logic circuitry, as well as any combinations of such components, embodied in programmers, such as physician or patient programmers, electrical stimulators, or other devices. The term “processor” or “processing circuitry” may generally refer to any of the foregoing logic circuitry, alone or in combination with other logic circuitry, or any other equivalent circuitry.

In the example illustrated in FIG. 19, one or more computing devices 230A-230N are connected to network 222. Network 222 is coupled to database 250. Database 250 may be any of the databases described herein, such as the GDSC database, or any other suitable database. Computing devices 230A-230N may send requests and receive reports from the network 222 and database 250. Computing devices 230A-230N may be computers located in hospitals, clinics, doctors' offices or other medical facilities. A clinician using a remote computing devices 230A-230N may request a drug-response prediction report for a specific patient or group of patients the clinician is authorized to access. In some examples, the clinician or another user may request a response-prediction report that presents information that is predictive of a response to one or more particular drugs for an identified group of one or more patients based on drug-response outcomes and expression profiles of cell lines for which data is accumulated in the database 250. The algorithms and processes performed for generating a drug-response prediction report may be performed by server 224 after receiving appropriate data from the database 250. In some examples, a computer device 230A, for example, may interrogate database 250 and parse information from appropriate fields of the database. In other examples, computer device 230A may transfer packets of data to database 250 that request specific types of information from which database 250 provides the information back to computer device 230A.

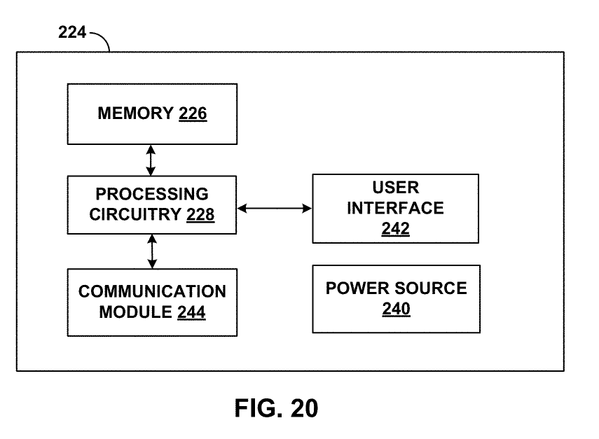

In some examples, an external server device, such as server device 224 shown in FIGS. 19 and 20, may also be connected to network 222. FIG. 20 is a functional block diagram of server 224, which may be used to implement the techniques described herein. In some examples, an external server device, such as server device 224, may also be connected to network 222. As shown in FIG. 20, server device 224 may include processing circuitry 228, memory 226, user interface 242, communication module 244, and power source 240. Processing circuitry 228 may include one or more processors. In one example, processing circuitry 228 is configured to run the software instructions in order to control operation of system 218. Processing circuitry 228 can include one or more processors, including one or more microprocessors, digital signal processors (DSPs), application specific integrated circuits (ASICs), field programmable gate arrays (FPGAs), or any other equivalent integrated or discrete logic circuitry, as well as any suitable combination of such components.

Memory 226 may include any volatile or non-volatile media, such as a random access memory (RAM), read only memory (ROM), non-volatile RAM (NVRAM), electrically erasable programmable ROM (EEPROM), flash memory, and the like. As mentioned above, memory 226 may store information including instructions for execution by processing circuitry 228 such as, but not limited to, instructions for performing the techniques described herein. Communication module 244 may provide one or more channels for receiving and/or transmitting information. Communication module 244 may be configured to perform wired and/or wireless communication with other devices, such as radio frequency communications. In other examples, communication module 244 may not be implemented, and instead, memory 226 may be removable (e.g., a removable flash memory).

Power source 240 delivers operating power to various components of computing device 218. Power source 240 may generate operational power from an alternating current source (e.g., residential or commercial electrical power outlet) or direct current source such as a rechargeable or non-rechargeable battery and a power generation circuit to produce the operating power. In other examples, non-rechargeable storage devices may be used for a limited period of time.

In one or more examples, the functions described in this disclosure may be implemented in hardware, software, firmware, or any combination thereof If implemented in software, the functions may be stored on, as one or more instructions or code, a computer-readable medium and executed by a hardware-based processing unit. Computer-readable media may include computer-readable storage media forming a tangible, non-transitory medium. Instructions may be executed by one or more processing circuitries, such as one or more DSPs, ASICs, FPGAs, general purpose microprocessors, or other equivalent integrated or discrete logic circuitry. Accordingly, the term “processing circuitry,” as used herein may refer to one or more of any of the foregoing structure or any other structure suitable for implementation of the techniques described herein.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, which may include various pieces of laboratory and computing equipment.

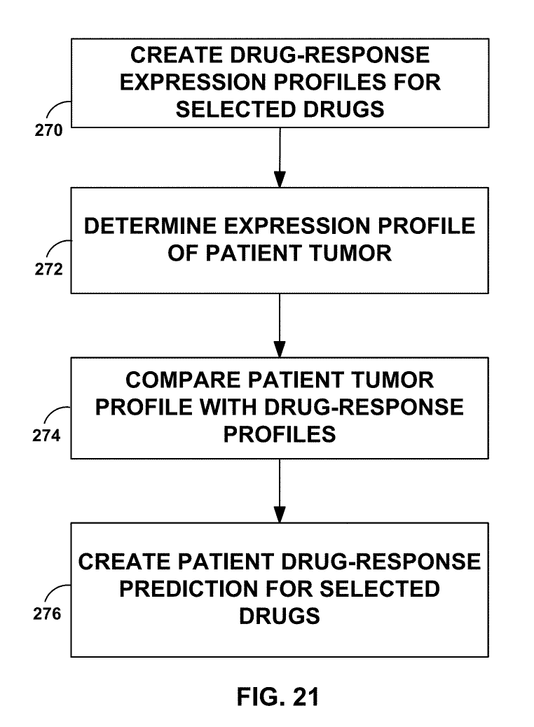

FIG. 21 is a flow diagram illustrating an example technique for creating a patient-specific drug-response prediction for one or more selected drugs. Server 224 will be described for the example of FIG. 21, but other devices, processing circuitry, or combination thereof, may be used to perform the technique of FIG. 21 and other techniques described herein in other examples.

According to the example of FIG. 21, processing circuitry 228 of server 224 may create drug-response expression profiles for one or more selected drugs. For example, server 224 may receive a request via network 222 from any of computing devices 230A-230N to create drug-response expression profiles for one or more drugs used in the treatment of B-cell malignancies. Server 224 may then access database 250 via network 222 to retrieve expression data and drug-response data for one or more malignant B-cell lines. For example, server 224 may extract certain data from database 250 related to specific drugs of interest or other various aspects of the database. Processing circuitry 228 then may analyze the expression and drug-response data for the malignant B-cell lines by any of the techniques described herein to generate a drug-response expression profile indicative of significant response to each of the selected drugs. For example, this analysis may include one or more different statistical analyses performed on the retrieved data to identify the significant response. The drug-response expression profiles may be stored in memory 226 of server 224 (270).

In some examples, processing circuitry 228 of server 224 may analyze one or more drug-response expression profiles to produce a drug-response prediction or identify a genetic predisposition of a particular patient, as described below with respect to the additional steps of the method illustrated in FIG. 21. In other examples, however, processing circuitry 228 may store the drug-response expression profiles in memory 226 for further analysis or future reference, thus concluding the method of FIG. 21 at (270). For example, such stored drug-response expression profiles may serve as a catalog of expression profiles that a clinician or other user later may use as a resource to identify treatment options for patients having gene-expression profiles that may be similar to one or more gene-expression profiles associated with a particular drug-response expression profile stored in memory 226.

In examples in which processing circuitry 228 continues to analyze the one or more drug-response expression profiles, server 224 may determine tumor expression profiles of tumors from one or more patients via data source 220. In some examples, server 224 may request that data source 220 generates the tumor expression profiles. Server 224 may store the tumor expression profiles in memory 226 (272). Server 224 then receives a request for a drug-response prediction report for a specific patient. Processing circuitry 228 of server 224 then compares the tumor expression profile of the patient to the drug-response expression profiles stored in memory 226 (274) and generates a patient-specific drug-response prediction that indicates drugs to which the patient is likely to respond (276). In some examples, processing circuitry 228 may produce a report of the drug-response prediction. In some such examples, the report of the drug-response prediction indicates an efficacious therapeutic effect of the drug on the patient tumor. In other examples, the report of the drug-response prediction indicates a non-efficacious effect of the drug on the patient tumor. In any such examples, processing circuitry 228 may store the report of the drug-response prediction in memory 226. In some examples, the patient-specific drug-response prediction also may indicate drugs to which the patient is unlikely to respond. Server 224 may employ one or more statistical techniques to determine drug-response prediction of drugs for the specific patient, in some examples. Server 224 then may transmit the patient-specific drug-response prediction back to one of computing devices 230A-230N. Finally, using this information, a clinician may select a drug to administer to the patient based on the patient-specific drug-response prediction.

In some examples of the method of FIG. 21 in which in which processing circuitry 228 generates drug-response expression profiles for more than one selected drug, processing circuitry 228 may interpret the request for the report of the drug-response prediction as being a plurality of requests for a corresponding plurality of drug-response predictions, such as a first request for a first drug-response prediction and a second request for a second drug-response prediction. For example, after generating a first drug-response prediction, processing circuitry 228 may receive, via network 222 from any of computing devices 230A-230N, a request for a report of a second drug-response prediction of the patient tumor to a second drug.

Responsive to the request, processing circuitry 228 may retrieve, from database 250, gene-expression data for a second plurality of cell lines and drug-response data indicating a response of each cell line of the second plurality of cell lines to the second drug. Next, processing circuitry 228 may generate a second drug-response expression profile based on the gene-expression data for the second plurality of cell lines and the drug-response data indicating a response of each cell line of the second plurality of cell lines to the second drug (270). In some examples, the second drug-response expression profile comprises a pattern of gene expression corresponding to an efficacious therapeutic effect of the second drug.

Processing circuitry 228 then may compare the gene-expression profile of the patient tumor to the second drug-response expression profile (274), determine whether the gene-expression profile of the patient tumor is substantially similar to the second drug-response expression profile, and produce the report of the second drug-response prediction (276). Processing circuitry 228 then may cause server 224 to transmit the second drug-response prediction back to one of computing devices 230A-230N. In some examples, processing circuitry 228 may produce a report of the drug-response prediction. As with other reports of a drug-response prediction (e.g., a first drug-response prediction), the report of the second drug-response prediction may an efficacious therapeutic effect of the drug on the patient tumor. In other examples, the report of the second drug-response prediction may indicate a non-efficacious effect of the drug on the patient tumor. In any such examples, processing circuitry 228 may store the report of the drug-response prediction in memory 226. In addition, in any such examples, the method of FIG. 21 may further include administering the drug to the patient having the patient tumor based on determining the report of the drug-response prediction.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various aspects of the disclosure have been described. These and other aspects are within the scope of the following claims.

What is claimed is:

1. A system comprising:
a memory; and
processing circuitry coupled to a communication network and configured to:
receive, from a remote computer, a gene-expression profile of a patient tumor;
receive, from the remote computer, a request for a report of a drug-response prediction of the patient tumor to a drug; and
responsive to the request, retrieve, from a database coupled to the communication network, gene-expression data for a plurality of cell lines and drug-response data indicating a response of each cell line of the plurality of cell lines to the drug;
generate a drug-response expression profile based on the gene-expression data for the plurality of cell lines and the drug-response data, wherein the drug-response expression profile comprises a pattern of gene expression corresponding to an efficacious therapeutic effect of the drug;
compare the gene-expression profile of the patient tumor to the drug-response expression profile;
determine whether the gene-expression profile of the patient tumor is substantially similar to the drug-response expression profile;
produce the report of the drug-response prediction;
store the report of the drug-response prediction in the memory; and
transmit the report of the drug-response prediction to the remote computer.

2. The system of claim 1, wherein the report of the drug-response prediction indicates an efficacious therapeutic effect of the drug on the patient tumor.

3. The system of claim 1, wherein the report of the drug-response prediction indicates a non-efficacious effect of the drug on the patient tumor.

4. The system of claim 1, wherein the drug comprises a first drug and the plurality of cell lines comprises a first plurality of cell lines, and wherein the processing circuitry is further configured to:
receive, from the remote computer, a request for a report of a second drug-response prediction of the patient tumor to a second drug;
responsive to the request, retrieve, from the database, gene-expression data for a second plurality of cell lines and drug-response data indicating a response of each cell line of the second plurality of cell lines to the second drug;
generate a second drug-response expression profile based on the gene-expression data for the second plurality of cell lines and the drug-response data indicating a response of each cell line of the second plurality of cell lines to the second drug, wherein the second drug-response expression profile comprises a pattern of gene expression corresponding to an efficacious therapeutic effect of the second drug;
compare the gene-expression profile of the patient tumor to the second drug-response expression profile;
determine whether the gene-expression profile of the patient tumor is substantially similar to the second drug-response expression profile;
produce the report of the second drug-response prediction;
store the report of the second drug-response prediction in the memory; and
transmit the report of the second drug-response prediction to the remote computer.

5. The system of claim 4, wherein the report of the second drug-response prediction indicates an efficacious therapeutic effect of the second drug on the patient tumor.

6. The system of claim 4, wherein the report of the second drug-response prediction indicates a non-efficacious effect of the second drug on the patient tumor.

7. The system of claim 1, wherein the efficacious therapeutic effect of the drug comprises at least one of a reduction in a size of the patient tumor, a change in one or more biomarkers indicative of a disease status, or a reduction in one or more patient symptoms.

8. A method, comprising:
receiving, by processing circuitry coupled to a communication network, a gene-expression profile of a patient tumor from a remote computer;
receiving, by the processing circuitry, a request for a report of a drug-response prediction of the patient tumor to a drug from the remote computer; and
responsive to the request:
retrieving, by the processing circuitry and from a database coupled to the communication network, gene-expression data for a plurality of cell lines and drug-response data indicating a response of each cell line of the plurality of cell lines to the drug;
generating, by the processing circuitry, a drug-response expression profile based on the gene-expression data for the plurality of cell lines and the drug-response data, wherein the drug-response expression profile comprises a pattern of gene expression corresponding to an efficacious therapeutic effect of the drug;
comparing, by the processing circuitry, the gene-expression profile of the patient tumor to the drug-response expression profile;
determining, by the processing circuitry, whether the gene-expression profile of the patient tumor is substantially similar to the drug-response expression profile;
producing, by the processing circuitry, the report of the drug-response prediction;
storing, by the processing circuitry, the report of the drug-response prediction in a memory; and
transmitting, by the processing circuitry, the report of the drug-response prediction to the remote computer.

9. The method of claim 8, wherein the report of the drug-response prediction indicates an efficacious therapeutic effect of the drug on the patient tumor.

10. The method of claim 8, wherein the report of the drug-response prediction indicates a non-efficacious effect of the drug on the patient tumor.

11. The method of claim 10, further comprising administering the drug to a patient having the patient tumor based on determining the report of the drug-response prediction.

12. The method of claim 8, wherein the drug comprises a first drug and the plurality of cell lines comprises a first plurality of cell lines, the method of claim 8 further comprising:
receiving, by the processing circuitry, a request for a report of a second drug-response prediction of the patient tumor to a second drug from the remote computer; and
responsive to the request:
retrieving, by the processing circuitry and from the database, gene-expression data for a second plurality of cell lines and drug-response data indicating a response of each cell line of the second plurality of cell lines to the second drug;
generating, by the processing circuitry, a second drug-response expression profile based on the gene-expression data for the second plurality of cell lines and the drug-response data indicating a response of each cell line of the second plurality of cell lines to the second drug, wherein the second drug-response expression profile comprises a pattern of gene expression corresponding to an efficacious therapeutic effect of the second drug;
comparing, by the processing circuitry, the gene-expression profile of the patient tumor to the second drug-response expression profile;
determining, by the processing circuitry, whether the gene-expression profile of the patient tumor is substantially similar to the second drug-response expression profile;
producing, by the processing circuitry, the report of the second drug-response prediction;
storing, by the processing circuitry, the report of the second drug-response prediction in the memory; and
transmitting, by the processing circuitry, the report of the second drug-response prediction to the remote computer.

13. The method of claim 12, wherein the report of the second drug-response prediction indicates an efficacious therapeutic effect of the second drug on the patient tumor.

14. The method of claim 12, wherein the report of the second drug-response prediction indicates a non-efficacious effect of the second drug on the patient tumor.

15. The method of claim 8, wherein the efficacious therapeutic effect of the drug comprises at least one of a reduction in a size of the patient tumor, a change in one or more biomarkers indicative of a disease status, or a reduction in one or more patient symptoms.

16. A non-transitory computer-readable storage medium comprising instructions that, when executed by processing circuitry, cause the processing circuitry to:
receive, from a remote computer, a gene-expression profile of a patient tumor;
receive, from the remote computer, a request for a report of a drug-response prediction of the patient tumor to a drug; and
responsive to the request:
retrieve, from a database coupled to a communication network, gene-expression data for a plurality of cell lines and drug-response data indicating a response of each cell line of the plurality of cell lines to a drug;
generate a drug-response expression profile based on the gene-expression data for the plurality of cell lines and the drug-response data, wherein the drug-response expression profile comprises a pattern of gene expression corresponding to an efficacious therapeutic effect of the drug;
compare the gene-expression profile of the patient tumor to the drug-response expression profile;
determine whether the gene-expression profile of the patient tumor is substantially similar to the drug-response expression profile;
produce the report of the drug-response prediction;
store the report of the drug-response prediction in a memory; and
transmit the report of the drug-response prediction to the remote computer.

17. The non-transitory computer-readable storage medium of claim 16, wherein the report of the drug-response prediction indicates an efficacious therapeutic effect of the drug on the patient tumor.

18. The non-transitory computer-readable storage medium of claim 16, wherein the report of the drug-response prediction indicates a non-efficacious effect of the drug on the patient tumor.

19. The non-transitory computer-readable storage medium of claim 16, wherein the efficacious therapeutic effect of the drug comprises at least one of a reduction in a size of the patient tumor, a change in one or more biomarkers indicative of a disease status, or a reduction in one or more patient symptoms.

20. The non-transitory computer-readable storage medium of claim 16, wherein the drug comprises a first drug and the plurality of cell lines comprises a first plurality of cell lines, and wherein the instructions, when executed by processing circuitry, further cause the processing circuitry to:
receive, from the remote computer, a request for a report of a second drug-response prediction of the patient tumor to a second drug;
responsive to the request, retrieve, from the database, gene-expression data for a second plurality of cell lines and drug-response data indicating a response of each cell line of the second plurality of cell lines to the second drug;
generate a second drug-response expression profile based on the gene-expression data for the second plurality of cell lines and the drug-response data indicating a response of each cell line of the second plurality of cell lines to the second drug, wherein the second drug-response expression profile comprises a pattern of gene expression corresponding to an efficacious therapeutic effect of the second drug;
compare the gene-expression profile of the patient tumor to the second drug-response expression profile;
determine whether the gene-expression profile of the patient tumor is substantially similar to the second drug-response expression profile;
produce the report of the second drug-response prediction;
store the report of the second drug-response prediction in the memory; and
transmit the report of the second drug-response prediction to the remote computer.

21. The non-transitory computer-readable storage medium of claim 20, wherein the report of the second drug-response prediction indicates an efficacious therapeutic effect of the second drug on the patient tumor.

22. The non-transitory computer-readable storage medium of claim 20, wherein the report of the second drug-response prediction indicates a non-efficacious effect of the second drug on the patient tumor.
